SEQUENCE LISTING (158 sequences)

SEQ ID NO: 1 - protein, 8 aa, Artificial Sequence
  Amino acid sequence of leader polypeptide 1
MDDDDDAA

SEQ ID NO: 2 - protein, 6 aa, Artificial Sequence
  Amino acid sequence of leader polypeptide 2
MDDDAA

SEQ ID NO: 3 - protein, 3 aa, Artificial Sequence
  Amino acid sequence of leader polypeptide 3
MDA

SEQ ID NO: 4 - protein, 9 aa, Artificial Sequence
  Amino acid sequence of leader polypeptide 4
MEEEEEEEE

SEQ ID NO: 5 - protein, 7 aa, Artificial Sequence
  Amino acid sequence of leader polypeptide 5
MEEEEEE

SEQ ID NO: 6 - protein, 5 aa, Artificial Sequence
  Amino acid sequence of leader polypeptide 6
MEEEE

SEQ ID NO: 7 - protein, 3 aa, Artificial Sequence
  Amino acid sequence of leader polypeptide 7
MEE

SEQ ID NO: 8 - protein, 3 aa, Artificial Sequence
  Amino acid sequence of leader polypeptide 8
MAE

SEQ ID NO: 9 - protein, 7 aa, Artificial Sequence
  Amino acid sequence of leader polypeptide 9
MCCCCCC

SEQ ID NO: 10 - protein, 4 aa, Artificial Sequence
  Amino acid sequence of leader polypeptide 10
MCCC

SEQ ID NO: 11 - protein, 3 aa, Artificial Sequence
  Amino acid sequence of leader polypeptide 11
MAC

SEQ ID NO: 12 - protein, 3 aa, Artificial Sequence
  Amino acid sequence of leader polypeptide 12
MAY

SEQ ID NO: 13 - protein, 3 aa, Artificial Sequence
  Amino acid sequence of leader polypeptide 13
MAA

SEQ ID NO: 14 - protein, 3 aa, Artificial Sequence
  Amino acid sequence of leader polypeptide 14
MGG

SEQ ID NO: 15 - protein, 4 aa, Artificial Sequence
  Amino acid sequence of leader polypeptide 15
MAKD

SEQ ID NO: 16 - protein, 4 aa, Artificial Sequence
  Amino acid sequence of leader polypeptide 16
MAKE

SEQ ID NO: 17 - protein, 3 aa, Artificial Sequence
  Amino acid sequence of leader polypeptide 17
MCH

SEQ ID NO: 18 - protein, 3 aa, Artificial Sequence
  Amino acid sequence of leader polypeptide 18
MAH

SEQ ID NO: 19 - protein, 5 aa, Artificial Sequence
  Amino acid sequence of leader polypeptide 19
MAHHH

SEQ ID NO: 20 - protein, 7 aa, Artificial Sequence
  Amino acid sequence of leader polypeptide 20
MAHHHHH

SEQ ID NO: 21 - protein, 4 aa, Artificial Sequence
  Amino acid sequence of leader polypeptide 21
MAKC

SEQ ID NO: 22 - protein, 3 aa, Artificial Sequence
  Amino acid sequence of leader polypeptide 22
MKY

SEQ ID NO: 23 - protein, 3 aa, Artificial Sequence
  Amino acid sequence of leader polypeptide 23
MAK

SEQ ID NO: 24 - protein, 4 aa, Artificial Sequence
  Amino acid sequence of leader polypeptide 24
MKAK

SEQ ID NO: 25 - protein, 5 aa, Artificial Sequence
  Amino acid sequence of leader polypeptide 25
MKKAK

SEQ ID NO: 26 - protein, 6 aa, Artificial Sequence
  Amino acid sequence of leader polypeptide 26
MKKKAK

SEQ ID NO: 27 - protein, 7 aa, Artificial Sequence
  Amino acid sequence of leader polypeptide 27
MKKKKAK

SEQ ID NO: 28 - protein, 8 aa, Artificial Sequence
  Amino acid sequence of leader polypeptide 28
MKKKKKAK

SEQ ID NO: 29 - protein, 4 aa, Artificial Sequence
  Amino acid sequence of leader polypeptide 29
MRAK

SEQ ID NO: 30 - protein, 5 aa, Artificial Sequence
  Amino acid sequence of leader polypeptide 30
MRRAK

SEQ ID NO: 31 - protein, 7 aa, Artificial Sequence
  Amino acid sequence of leader polypeptide 31
MRRRRAK

SEQ ID NO: 32 - protein, 9 aa, Artificial Sequence
  Amino acid sequence of leader polypeptide 32
MRRRRRRAK

SEQ ID NO: 33 - protein, 11 aa, Artificial Sequence
  Amino acid sequence of leader polypeptide 33
MRRRRRRRRAK

SEQ ID NO: 34 - DNA, 48 bp, Artificial Sequence
  Forward primer for leader polypeptide 1
catatggacgatgacgatgacgctgcaccgtcttatccgccaacctac

SEQ ID NO: 35 - DNA, 42 bp, Artificial Sequence
  Forward primer for leader polypeptide 2
catatggacgatgacgctgcaccgtcttatccgccaacctac

SEQ ID NO: 36 - DNA, 33 bp, Artificial Sequence
  Forward primer for leader polypeptide 3
catatggacgctccgtcttatccgccaacctac

SEQ ID NO: 37 - DNA, 51 bp, Artificial Sequence
  Forward primer for leader polypeptide 4
catatggaagaggaagaggaagaggaagagccgtcttatccgccaacctac

SEQ ID NO: 38 - DNA, 45 bp, Artificial Sequence
  Forward primer for leader polypeptide 5
catatggaagaggaagaggaagagccgtcttatccgccaacctac

SEQ ID NO: 39 - DNA, 39 bp, Artificial Sequence
  Forward primer for leader polypeptide 6
catatggaagaggaagagccgtcttatccgccaacctac

SEQ ID NO: 40 - DNA, 33 bp, Artificial Sequence
  Forward primer for leader polypeptide 7
catatggaagagccgtcttatccgccaacctac

SEQ ID NO: 41 - DNA, 33 bp, Artificial Sequence
  Forward primer for leader polypeptide 8
catatggctgaaccgtcttatccgccaacctac

SEQ ID NO: 42 - DNA, 45 bp, Artificial Sequence
  Forward primer for leader polypeptide 9
catatgtgctgttgctgttgctgtccgtcttatccgccaacctac

SEQ ID NO: 43 - DNA, 36 bp, Artificial Sequence
  Forward primer for leader polypeptide 10
catatgtgctgttgcccgtcttatccgccaacctac

SEQ ID NO: 44 - DNA, 33 bp, Artificial Sequence
  Forward primer for leader polypeptide 11
catatggcttgcccgtcttatccgccaacctac

SEQ ID NO: 45 - DNA, 33 bp, Artificial Sequence
  Forward primer for leader polypeptide 12
catatggcttacccgtcttatccgccaacctac

SEQ ID NO: 46 - DNA, 33 bp, Artificial Sequence
  Forward primer for leader polypeptide 13
catatggctgcaccgtcttatccgccaacctac

SEQ ID NO: 47 - DNA, 33 bp, Artificial Sequence
  Forward primer for leader polypeptide 14
catatgggtggtccgtcttatccgccaacctac

SEQ ID NO: 48 - DNA, 36 bp, Artificial Sequence
  Forward primer for leader polypeptide 15
catatggctaaagacccgtcttatccgccaacctac

SEQ ID NO: 49 - DNA, 36 bp, Artificial Sequence
  Forward primer for leader polypeptide 16
catatggctaaagaaccgtcttatccgccaacctac

SEQ ID NO: 50 - DNA, 33 bp, Artificial Sequence
  Forward primer for leader polypeptide 17
catatgtgccacccgtcttatccgccaacctac

SEQ ID NO: 51 - DNA, 33 bp, Artificial Sequence
  Forward primer for leader polypeptide 18
catatggctcacccgtcttatccgccaacctac

SEQ ID NO: 52 - DNA, 39 bp, Artificial Sequence
  Forward primer for leader polypeptide 19
catatggctcaccatcacccgtcttatccgccaacctac

SEQ ID NO: 53 - DNA, 45 bp, Artificial Sequence
  Forward primer for leader polypeptide 20
catatggctcaccatcaccatcacccgtcttatccgccaacctac

SEQ ID NO: 54 - DNA, 36 bp, Artificial Sequence
  Forward primer for leader polypeptide 21
catatggctaaatgcccgtcttatccgccaacctac

SEQ ID NO: 55 - DNA, 33 bp, Artificial Sequence
  Forward primer for leader polypeptide 22
catatgaaatacccgtcttatccgccaacctac

SEQ ID NO: 56 - DNA, 33 bp, Artificial Sequence
  Forward primer for leader polypeptide 23
catatggctaagccgtcttatccgccaacctac

SEQ ID NO: 57 - DNA, 36 bp, Artificial Sequence
  Forward primer for leader polypeptide 24
catatgaaagctaagccgtcttatccgccaacctac

SEQ ID NO: 58 - DNA, 39 bp, Artificial Sequence
  Forward primer for leader polypeptide 25
catatgaaaaaagctaagccgtcttatccgccaacctac

SEQ ID NO: 59 - DNA, 42 bp, Artificial Sequence
  Forward primer for leader polypeptide 26
catatgaaaaaaaaagctaagccgtcttatccgccaacctac

SEQ ID NO: 60 - DNA, 45 bp, Artificial Sequence
  Forward primer for leader polypeptide 27
catatgaaaaaaaaaaaagctaagccgtcttatccgccaacctac

SEQ ID NO: 61 - DNA, 48 bp, Artificial Sequence
  Forward primer for leader polypeptide 28
catatgaaaaaaaaaaaaaaagctaagccgtcttatccgccaacctac

SEQ ID NO: 62 - DNA, 36 bp, Artificial Sequence
  Forward primer for leader polypeptide 29
catatgagagctaagccgtcttatccgccaacctac

SEQ ID NO: 63 - DNA, 36 bp, Artificial Sequence
  Forward primer for leader polypeptide 30
catatgcgtcgcgctaagccgtcttatccgccaacc

SEQ ID NO: 64 - DNA, 42 bp, Artificial Sequence
  Forward primer for leader polypeptide 31
catatgcgtcgccgtcgcgctaagccgtcttatccgccaacc

SEQ ID NO: 65 - DNA, 48 bp, Artificial Sequence
  Forward primer for leader polypeptide 32
catatgcgtcgccgtcgccgtcgcgctaagccgtcttatccgccaacc

SEQ ID NO: 66 - DNA, 54 bp, Artificial Sequence
  Forward primer for leader polypeptide 33
catatgcgtcgccgtcgccgtcgccgtcgcgctaagccgtcttatccgccaacc

SEQ ID NO: 67 - DNA, 21 bp, Artificial Sequence
  Reverse primer for leader polypeptide form 1 to 33
ctcgaggtcgacaagcttacg

SEQ ID NO: 68 - protein, 21 aa, Artificial Sequence
  Anticipated amino acid sequence of PhoA
MKQSTIALALLPLLFTPVTKA

SEQ ID NO: 69 - protein, 21 aa, Artificial Sequence
  Anticipated amino acid sequence of OmpA
MKKTAIAIAVALAGFATVAQA

SEQ ID NO: 70 - protein, 23 aa, Artificial Sequence
  Anticipated amino acid sequence of StII
MKKNIAFLLASMFVFSIATNAYA

SEQ ID NO: 71 - protein, 21 aa, Artificial Sequence
  Anticipated amino acid sequence of PhoE
MKKSTLALVVMGIVASASVQA

SEQ ID NO: 72 - protein, 26 aa, Artificial Sequence
  Anticipated amino acid sequence of MalE
MKIKTGARILALSALTTMMFSASALA

SEQ ID NO: 73 - protein, 21 aa, Artificial Sequence
  Anticipated amino acid sequence of OmpC
MKVKVLSLLVPALLVAGAANA

SEQ ID NO: 74 - protein, 20 aa, Artificial Sequence
  Anticipated amino acid sequence of Lpp
MKATKLVLGAVILGSTLLAG

SEQ ID NO: 75 - protein, 21 aa, Artificial Sequence
  Anticipated amino acid sequence of LTB
MNKVKCYVLFTALLSSLYAHG

SEQ ID NO: 76 - protein, 22 aa, Artificial Sequence
  Anticipated amino acid sequence of OmpF
MMKRNILAVIVPALLVAGTANA

SEQ ID NO: 77 - protein, 25 aa, Artificial Sequence
  Anticipated amino acid sequence of LamB
MMITLRKLPLAVAVAAGVMSAQAMA

SEQ ID NO: 78 - protein, 20 aa, Artificial Sequence
  Anticipated amino acid sequence of OmpT
MRAKLLGIVLTTPIAISSFA

SEQ ID NO: 79 - protein, 33 aa, Artificial Sequence
  Anticipated amino acid sequence of FdnG
MDVSRRQFFKICAGGMAGTTVAALGFAPKQALA

SEQ ID NO: 80 - protein, 33 aa, Artificial Sequence
  Anticipated amino acid sequence of FdoG
MQVSRRQFFKICAGGMAGTTAAALGFAPSVALA

SEQ ID NO: 81 - protein, 41 aa, Artificial Sequence
  Anticipated amino acid sequence of NapG
MSRSAKPQNGRRRFLRDVVRTAGGLAAVGVALGLQQQTARA

SEQ ID NO: 82 - protein, 45 aa, Artificial Sequence
  Anticipated amino acid sequence of HyaA
MNNEETFYQAMRRQGVTRRSFLKYCSLAATSLGLGAGMAPKIAWA

SEQ ID NO: 83 - protein, 42 aa, Artificial Sequence
  Anticipated amino acid sequence of YnfE
MSKNERMVGISRRTLVKSTAIGSLALAAGGFSLPFTLRNAAA

SEQ ID NO: 84 - protein, 28 aa, Artificial Sequence
  Anticipated amino acid sequence of WcaM
MPFKKLSRRTFLTASSALAFLHTPFARA

SEQ ID NO: 85 - protein, 42 aa, Artificial Sequence
  Anticipated amino acid sequence of TorA
MNNNDLFQASRRRFLAQLGGLTVAGMLGPSLLTPRRATAAQA

SEQ ID NO: 86 - protein, 31 aa, Artificial Sequence
  Anticipated amino acid sequence of NapA
MKLSRRSFMKANAVAAAAAAAGLSVPGVARA

SEQ ID NO: 87 - protein, 30 aa, Artificial Sequence
  Anticipated amino acid sequence of YcbK
MDKFDANRRKLLALGGVALGAAILPTPAFA

SEQ ID NO: 88 - protein, 45 aa, Artificial Sequence
  Anticipated amino acid sequence of DmsA
MKTKIPDAVLAAEVSRRGLVKTTAIGGLAMASSALTLPFSRIAHA

SEQ ID NO: 89 - protein, 33 aa, Artificial Sequence
  Anticipated amino acid sequence of YahJ
MKESNSRREFLSQSGKMVTAAALFGTSVPLAHA

SEQ ID NO: 90 - protein, 44 aa, Artificial Sequence
  Anticipated amino acid sequence of YedY
MKKNQFLKESDVTAESVFFMKRRQVLKALGISATALSLPHAAHA

SEQ ID NO: 91 - protein, 27 aa, Artificial Sequence
  Anticipated amino acid sequence of SufI
MSLSRRQFIQASGIALCAGAVPLKASA

SEQ ID NO: 92 - protein, 35 aa, Artificial Sequence
  Anticipated amino acid sequence of YcdB
MQYKDENGVNEPSRRRLLKVIGALALAGSCPVAHA

SEQ ID NO: 93 - protein, 37 aa, Artificial Sequence
  Anticipated amino acid sequence of TorZ
MIREEVMTLTRREFIKHSGIAAGALVVTSAAPLPAWA

SEQ ID NO: 94 - protein, 28 aa, Artificial Sequence
  Anticipated amino acid sequence of HybA
MNRRNFIKAASCGALLTGALPSVSHAAA

SEQ ID NO: 95 - protein, 49 aa, Artificial Sequence
  Anticipated amino acid sequence of YnfF
MMKIHTTEALMKAEISRRSLMKTSALGSLALASSAFTLPFSQMVRAAEA

SEQ ID NO: 96 - protein, 37 aa, Artificial Sequence
  Anticipated amino acid sequence of HybO
MTGDNTLIHSHGINRRDFMKLCAALAATMGLSSKAAA

SEQ ID NO: 97 - protein, 34 aa, Artificial Sequence
  Anticipated amino acid sequence of AmiA
MSTFKPLKTLTSRRQVLKAGLAALTLSGMSQAIA

SEQ ID NO: 98 - protein, 32 aa, Artificial Sequence
  Anticipated amino acid sequence of MdoD
MDRRRFIKGSMAMAAVCGTSGIASLFSQAAFA

SEQ ID NO: 99 - protein, 30 aa, Artificial Sequence
  Anticipated amino acid sequence of FhuD
MSGLPLISRRRLLTAMALSPLLWQMNTAHA

SEQ ID NO: 100 - protein, 26 aa, Artificial Sequence
  Anticipated amino acid sequence of YcdO
MTINFRRNALQLSVAALFSSAFMANA

SEQ ID NO: 101 - protein, 16 aa, Artificial Sequence
  Amino acid sequence of leader polypeptide 101
MEETAIAIRRRRRRRR

SEQ ID NO: 102 - protein, 16 aa, Artificial Sequence
  Amino acid sequence of leader polypeptide 102
MAATAIAIRRRRRRRR

SEQ ID NO: 103 - protein, 16 aa, Artificial Sequence
  Amino acid sequence of leader polypeptide 103
MAHTAIAIRRRRRRRR

SEQ ID NO: 104 - protein, 16 aa, Artificial Sequence
  Amino acid sequence of leader polypeptide 104
MKKTAIAIRRRRRRRR

SEQ ID NO: 105 - protein, 16 aa, Artificial Sequence
  Amino acid sequence of leader polypeptide 105
MRRTAIAIRRRRRRRR

SEQ ID NO: 106 - protein, 7 aa, Artificial Sequence
  Amino acid sequence of leader polypeptide 106
MDDDDDD

SEQ ID NO: 107 - protein, 7 aa, Artificial Sequence
  Amino acid sequence of leader polypeptide 107
MEEEEEE

SEQ ID NO: 108 - protein, 7 aa, Artificial Sequence
  Amino acid sequence of leader polypeptide 108
MKKKKKK

SEQ ID NO: 109 - protein, 7 aa, Artificial Sequence
  Amino acid sequence of leader polypeptide 109
MRRRRRR

SEQ ID NO: 110 - protein, 10 aa, Artificial Sequence
  Amino acid sequence of leader polypeptide 110
MRRRRRRRRR

SEQ ID NO: 111 - protein, 13 aa, Artificial Sequence
  Amino acid sequence of leader polypeptide 111
MRRRRRRRRRRRR

SEQ ID NO: 112 - protein, 7 aa, Artificial Sequence
  Amino acid sequence of leader polypeptide 112
MKKRKKR

SEQ ID NO: 113 - protein, 7 aa, Artificial Sequence
  Amino acid sequence of leader polypeptide 113
MKKRKKR

SEQ ID NO: 114 - protein, 7 aa, Artificial Sequence
  Amino acid sequence of leader polypeptide 114
MRRKRRK

SEQ ID NO: 115 - protein, 7 aa, Artificial Sequence
  N-terminal(1-7) of GFP
MVSKGEE

SEQ ID NO: 116 - protein, 7 aa, Artificial Sequence
  N-terminal(1-7) of GFP variant(V2E)
MESKGEE

SEQ ID NO: 117 - protein, 7 aa, Artificial Sequence
  N-terminal(1-7) of GFP variant(V2E-S3E)
MEEKGEE

SEQ ID NO: 118 - protein, 7 aa, Artificial Sequence
  N-terminal(1-7) of GFP variant(V2E-S3E-K4E)
MEEEGEE

SEQ ID NO: 119 - protein, 7 aa, Artificial Sequence
  N-terminal(1-7) of GFP variant(V2E-S3E-K4E-G5E)
MEEEEEE

SEQ ID NO: 120 - protein, 25 aa, Artificial Sequence
  TorAss-GFP(1-7)
MLGPSLLTPRRATAAQAAMVSKGEE

SEQ ID NO: 121 - protein, 23 aa, Artificial Sequence
  MKK-OmpAss(4-23)
MKKTAIAIAVALAGFATVAQAAP

SEQ ID NO: 122 - protein, 27 aa, Artificial Sequence
  MKKKKKK-OmpAss(4-23)
MKKKKKKTAIAIAVALAGFATVAQAAP

SEQ ID NO: 123 - DNA, 72 bp, Artificial Sequence
  Forward primer for Seq ID No. 101
catatggaagagacagctatcgcgattcgccgtcgccgtcgccgtcgccgtatggtgagcaagggcgaggag

SEQ ID NO: 124 - DNA, 72 bp, Artificial Sequence
  Forward primer for Seq ID No. 102
catatggctgcaacagctatcgcgattcgccgtcgccgtcgccgtcgccgtatggtgagcaagggcgaggag

SEQ ID NO: 125 - DNA, 72 bp, Artificial Sequence
  Forward primer for Seq ID No. 103
catatggctcacacagctatcgcgattcgccgtcgccgtcgccgtcgccgtatggtgagcaagggcgaggag

SEQ ID NO: 126 - DNA, 72 bp, Artificial Sequence
  Forward primer for Seq ID No. 104
catatgaaaaaaacagctatcgcgattcgccgtcgccgtcgccgtcgccgtatggtgagcaagggcgaggag

SEQ ID NO: 127 - DNA, 72 bp, Artificial Sequence
  Forward primer for Seq ID No. 105
catatgcgtcgcacagctatcgcgattcgccgtcgccgtcgccgtcgccgtatggtgagcaagggcgaggag

SEQ ID NO: 128 - DNA, 45 bp, Artificial Sequence
  Forward primer for Seq ID No. 106
catatggacgatgacgatgacgatatggtgagcaagggcgaggag

SEQ ID NO: 129 - DNA, 45 bp, Artificial Sequence
  Forward primer for Seq ID No. 107
catatggaagaagaagaagaagaaatggtgagcaagggcgaggag

SEQ ID NO: 130 - DNA, 45 bp, Artificial Sequence
  Forward primer for Seq ID No. 108
catatgaaaaaaaaaaaaaaaaaaatggtgagcaagggcgaggag

SEQ ID NO: 131 - DNA, 45 bp, Artificial Sequence
  Forward primer for Seq ID No. 109
catatgcgtcgccgtcgccgtcgcatggtgagcaagggcgaggag

SEQ ID NO: 132 - DNA, 54 bp, Artificial Sequence
  Forward primer for Seq ID No. 110
catatgcgtcgccgtcgccgtcgccgtcgccgtatggtgagcaagggcgaggag

SEQ ID NO: 133 - DNA, 63 bp, Artificial Sequence
  Forward primer for Seq ID No. 111
catatgcgtcgccgtcgccgtcgccgtcgccgtcgccgtcgcatggtgagcaagggcgaggag

SEQ ID NO: 134 - DNA, 45 bp, Artificial Sequence
  Forward primer for Seq ID No. 112
catatgaaaaaacgcaaaaaacgcatggtgagcaagggcgaggag

SEQ ID NO: 135 - DNA, 45 bp, Artificial Sequence
  Forward primer for Seq ID No. 113
catatgaagaaacgcaagaaacgcatggtgagcaagggcgaggag

SEQ ID NO: 136 - DNA, 45 bp, Artificial Sequence
  Forward primer for Seq ID No. 114
catatgcgtcgcaaacgtcgcaaaatggtgagcaagggcgaggag

SEQ ID NO: 137 - DNA, 24 bp, Artificial Sequence
  Forward primer for GFP
catatggtgagcaagggcgaggag

SEQ ID NO: 138 - DNA, 39 bp, Artificial Sequence
  Forward primer for GFP variant(V2E)
catatggaaagcaagggcgaggagctgttcaccggggtg

SEQ ID NO: 139 - DNA, 39 bp, Artificial Sequence
  Forward primer for GFP variant(V2E-S3E)
catatggaagaaaagggcgaggagctgttcaccggggtg

SEQ ID NO: 140 - DNA, 39 bp, Artificial Sequence
  Forward primer for GFP variant(V2E-S3E-K4E)
catatggaagaagaaggcgaggagctgttcaccggggtg

SEQ ID NO: 141 - DNA, 39 bp, Artificial Sequence
  Forward primer for GFP variant(V2E-S3E-K4E-G5E)
catatggaagaagaagaagaggagctgttcaccggggtg

SEQ ID NO: 142 - DNA, 90 bp, Artificial Sequence
  Primary forward primer for TorAss-GFP(1-7)
ttaaccgtcgccgggatgctggggccgtcattgttaacgccgcgacgtgcgactgcggcgcaagcggcgatggtgagcaagggcgaggag

SEQ ID NO: 143 - DNA, 84 bp, Artificial Sequence
  Secondary forward primer for TorAss-GFP(1-7)
catatgaacaataacgatctctttcaggcatcacgtcggcgttttcgtgcacaactcggcggcttaaccgtcgccgggatgctg

SEQ ID NO: 144 - DNA, 93 bp, Artificial Sequence
  Forward primer for MKK-OmpAss(4-23)
catatgaaaaagacagctatcgcgattgcagtggcactggctggtttcgctaccgtagcgcaggccgctccgatggtgagcaagggcgaggag

SEQ ID NO: 145 - DNA, 105 bp, Artificial Sequence
  Forward primer for MKKKKKK-OmpAss(4-23)
catatgaaaaaaaaaaaaaaaaaaacagctatcgcgattgcagtggcactggctggtttcgctaccgtagcgcaggccgctccgatggtgagcaagggcgaggag

SEQ ID NO: 146 - DNA, 27 bp, Artificial Sequence
  Reverse primer for GFP with leader polypeptides and variants thereof
ctcgagcttgtacagctcgtccatgcc

SEQ ID NO: 147 - protein, 6 aa, Artificial Sequence
  1st-6th amino acids of N-terminal of Pf3
MQSVIT

SEQ ID NO: 148 - protein, 7 aa, Artificial Sequence
  1st-7th amino acids of N-terminal of Pf3
MQSVITD

SEQ ID NO: 149 - protein, 3 aa, Artificial Sequence
  1st-3rd amino acids of N-terminal of M13 coat protein
MKK

SEQ ID NO: 150 - protein, 8 aa, Artificial Sequence
  1st-8th amino acids of N-terminal of M13 coat protein
MKKSLVLK

SEQ ID NO: 151 - protein, 3 aa, Artificial Sequence
  Synthetic signal peptide MRR
MRR

SEQ ID NO: 152 - protein, 16 aa, Artificial Sequence
  MAH-OmpSP4-10-6XArg
MAHTAIAIAVRRRRRR

SEQ ID NO: 153 - protein, 16 aa, Artificial Sequence
  MEE-OmpSP4-10-6XGlu
MEETAIAIAVEEEEEE

SEQ ID NO: 154 - protein, 10 aa, Artificial Sequence
  Mefp1
AKPSYPPTYK

SEQ ID NO: 155 - DNA, 53 bp, Artificial Sequence
  pET-22(+) and MKKKKKK-GFP1-5 encoding region
aagaaggagatatacatatgaaaaaaaaaaaaaaaaaaatggtgagcaagggc

SEQ ID NO: 156 - DNA, 53 bp, Artificial Sequence
  pET-22(+) and MRRRRRR-GFP1-5 encoding region
aagaaggagatatacatatgcgtcgccgtcgccgtcgcatggtgagcaagggc

SEQ ID NO: 157 - protein, 8 aa, Artificial Sequence
  Synthetic leader peptide MKKKKKKK
MKKKKKKK

SEQ ID NO: 158 - protein, 8 aa, Artificial Sequence
  Synthetic leader peptide MRRRRRRR
MRRRRRRR

(19) United States
(12) Patent Application Publication
(10) Pub. No.: US 2013/0084602 A1
(43) Pub. Date: Apr. 4, 2013

(54) SOLUBLE EXPRESSION OF BULKY FOLDED ACTIVE PROTEINS

(71) Applicants: Sang Jun Lee, Busan (KR); Young Ok Kim, Busan (KR); Bo Hye Nam, Busan (KR); Hee Jeong Kong, Busan (KR); Kyung Kil Kim, Busan (KR)

(72) Inventors: Sang Jun Lee, Busan (KR); Young Ok Kim, Busan (KR); Bo Hye Nam, Busan (KR); Hee Jeong Kong, Busan (KR); Kyung Kil Kim, Busan (KR)

(73) Assignee: Republic of Korea Represented by National Fisheries Research & Development Institute, Busan (KR)

(21) Appl. No.: 13/643,137

(22) PCT Filed: Mar. 3, 2011
(86) PCT No.: PCT/KR2011/001465

§ 371 Date: Oct. 24, 2012

(30) Foreign Application Priority Data
May 11, 2010 (KR) 10-2010-0043855

(51) Int. Cl. C12N 15/70; C12P 21/06

(52) U.S. Cl. 435/68.1; 435/320.1; 435/471

(57) ABSTRACT

The present invention relates to expression vectors and methods for enhancing soluble expression and secretion of a heterologous protein, particularly a bulky folded active heterologous protein which has one or more transmembrane-like domains or intramolecular disulfide bonds by linking a leader peptide with acidic or basic pI and high hydrophilicity thereto; by substituting one or more amino acids within N-terminal of the heterologous protein with ones having acidic or neutral pI and high hydrophilicity; or reducing elevating G_{RNA} value of a polynucleotide encoding the leader peptide having basic pI value and high hydrophilicity. The expression vector and the method may be used to produce of heterologous protein and to transduce of therapeutic proteins in a patient by preventing formation of insoluble inclusion body and by enhancing secretional efficiency of the heterologous protein into the periplasm or outside cell.

### TECHNICAL FIELD

The present invention relates to expression vectors and methods for enhancing the soluble expression of heterologous proteins in cytosol and the secretion thereof.

### BACKGROUND ART

The key point of current biotechnology is the production of heterologous proteins and particularly the production of soluble proteins in native form easily. The production of soluble proteins is important for the synthesis and the recovery of active proteins, the crystallization for functional researches, and the industrialization thereof. Until now many researches related to the production of recombinant heterologous proteins using E. coli. The reason why E. coli is used is that it has many benefits such as easy manipulation, its rapid growth rate, safe expression, low cost and relative convenience of scale-up.

However E. coli has no post-translation chaperons and post-translational processing, thus recombinant heterologous proteins expressed in E. coli are not folded properly or are formed as insoluble inclusion bodies (Baneyx, Curr. Opin.Biotechnol., 10: 411-421, 1999).

In order to solve these problems, researches on the structure and the function of signal sequences based on the fact that signal sequences make proteins be secreted into the periplasm and vectors for expressing soluble heterologous proteins have been developed using various signal sequences from the researches (Ghrayeb et al., EMBO J. 3: 2437-2442, 1984; Kohl et al., Nucleic Acids Res., 18: 1069, 1990; Morika-Fujimoto et al., J. Biol. Chem., 266: 1728-1732, 1991).

### SUMMARY OF INVENTION

### Technical Problem

However, previous expression vectors did not express bulky folded active proteins such as GFP (green fluorescent protein) well in soluble form, which have intramolecular one or more disulfide bonds or transmembrane domains.

Thus, the present invention is designed in order to solve many problems including these problems. The purpose of the present invention is to provide an expression vector for enhancing soluble expression and secretion of bulky folded active proteins having one or more inherent transmembrane-like domains or intramolecular disulfide bonds.

The other purpose of the present invention is to provide a method for enhancing soluble expression and secretion of bulky folded active proteins having one or more inherent transmembrane-like domains or intramolecular disulfide bonds.

However these technical problems are exemplified thus the scope of the present invention is not limited thereto.

### SOLUTION TO PROBLEM

According to an aspect of the present invention, an expression vector for enhancing soluble expression and secretion of bulky folded active heterologous proteins having one or more inherent transmembrane-like domains or intramolecular disulfide bonds, comprising a gene construct consisting of: 1) a promoter; and, 2) a polynucleotide operably linked to the promoter, encoding a leader peptide having N-terminal whose pI value is 2.00 to 9.60 and whose hydrophilicity is 1.00 to 2.00 is provided.

According to an aspect of the present invention, a gene construct consisting of: 1) a promoter; and, 2) a polynucleotide operably linked to the promoter, which encodes a leader peptide having N-terminal whose pI value is 2.00 to 9.60 and whose hydrophilicity is 1.00 to 2.00 is provided.

According to an aspect of the present invention, a method for enhancing soluble expression and secretion of a bulky folded active heterologous protein having one or more inherent transmembrane-like domains or intramolecular disulfide bonds comprising:

Providing a polynucleotide encoding a leader peptide having N-terminal whose pI value is 2.00 to 9.60 and whose hydrophilicity is 1.00 to 2.00;

Constructing a gene construct consisting of the polynucleotide and a polynucleotide encoding the bulky folded active heterologous protein having one or more inherent transmembrane-like domains or intramolecular disulfide bonds;

Constructing a recombinant expression vector by operably inserting the gene construct into an expression vector;

Producing transformants by transforming host cells with the recombinant expression vector; and,

Selecting a transformant whose ability for expressing and secreting the bulky folded active heterologous protein is good among the transformants is provided.

According to an aspect of the present invention, a method for producing a bulky folded active heterologous protein having one or more inherent transmembrane-like domains or intramolecular disulfide bonds comprising:

Providing a polynucleotide encoding a leader peptide having N-terminal whose pI value is 2.00 to 9.60 and whose hydrophilicity is 1.00 to 2.00;

Constructing a gene construct encoding a fusion protein sequentially consisting of the leader peptide, a protease recognition site and the bulky folded active heterologous protein having one or more inherent transmembrane-like domains or intramolecular disulfide bonds;

Constructing a recombinant expression vector by operably inserting the gene construct into an expression vector;

Producing transformants by transforming host cells with the recombinant expression vector; and,

Culturing the transformants by inoculating culture media with the transformants;

Isolating the fusion protein; and

Isolating a native form of the bulky folded active heterologous protein after cleaving the protease recognition site with a protease is provided.

According to an aspect of the present invention, an expression vector for enhancing soluble expression and secretion of bulky folded active heterologous proteins having one or more inherent transmembrane-like domains or intramolecular disulfide bonds, comprising a gene construct consisting of: 1) a promoter; and, 2) a polynucleotide operably linked to the promoter, encoding a leader peptide having N-terminal whose pI value is 9.90 to 13.35 and whose hydrophilicity is 1.00 to 2.50, wherein the polynucleotide has ΔG_{RNA} value of more than −10.00 is provided.

According to an aspect of the present invention, a gene construct consisting of: 1) a promoter; and, 2) a polynucleotide operably linked to the promoter, encoding a leader peptide having N-terminal whose pI value is 9.90 to 13.35 and whose hydrophilicity is 1.00 to 2.50, wherein the polynucleotide has ΔG_{RNA} value of more than −10.00 is provided.

According to another aspect of the present invention, a method for enhancing soluble expression and secretion of a bulky folded active heterologous protein having one or more inherent transmembrane-like domains or intramolecular disulfide bonds, the method comprising:

Providing a polynucleotide encoding a leader peptide having N-terminal whose pI value is 9.90 to 13.35 and whose hydrophilicity is 1.00 to 2.50, wherein the polynucleotide has ΔG_{RNA}value of more than −10.0;

Constructing a gene construct consisting of the polynucleotide and a polynucleotide encoding the bulky folded active heterologous protein having one or more inherent transmembrane-like domains or intramolecular disulfide bonds, wherein the bulky folded active heterologous protein moves into the periplasm as a folded form and has biological activity in the periplasm;

Constructing a recombinant expression vector by operably inserting the gene construct into an expression vector;

Producing transformants by transforming host cells with the recombinant expression vector; and,

Selecting a transformant whose ability for expressing and secreting the bulky folded active heterologous protein is good among the transformants is provided.

### BRIEF DESCRIPTION OF DRAWINGS

FIG. 1A is a photograph of Western blot of rMefp1 solubly expressed by N-terminal leader peptide having various pI value:

(a) M: marker, 1: MAK (SEQ ID No: 23), 2: MD₅AA (SEQ ID No: 1), 3: MD₃AA (SEQ ID No: 2), 4: MDA (SEQ ID No: 3), 5: ME₈(SEQ ID No: 4), 6: ME₆(SEQ ID No: 5), 7: ME₄ (SEQ ID No: 6), 8: ME₂(SEQ ID No: 7), and 9: MAE (SEQ ID No: 8);

(b) M: marker, 1: MAK (SEQ ID No: 23), 2: MC₆(SEQ ID No: 9), 3: MC₃(SEQ ID No: 10), 4: MAC (SEQ ID No: 11), 5: MAY (SEQ ID No: 12), 6: MAA (SEQ ID No: 13), 7: MGG (SEQ ID No: 14), 8: MAKD (SEQ ID No: 15), and 9: MAKE (SEQ ID No: 16);

(c) M: marker, 1: MAK (SEQ ID No: 23), 2: MCH (SEQ ID No: 17), 3: MAH (SEQ ID No: 18), 4: MAH₃(SEQ ID No: 19), 5: MAH₅(SEQ ID No: 20), 6: MAKC (SEQ ID No: 21), and 7: MKY (SEQ ID No: 22);

(d) M: marker, 1: MAK (SEQ ID No: 23), 2: MKAK (SEQ ID No: 24), 3: MK₂AK (SEQ ID No: 25), 4: MK₃AK (SEQ ID No: 26); 5: MK₄AK (SEQ ID No: 27), and 6: MK₅AK (SEQ ID No: 28); and

(e) M: marker, 1: MAK (SEQ ID No: 23), 2: MRAK (SEQ ID No: 29), 3: MR₂AK (SEQ ID No: 30), 4: MR₄AK (SEQ ID No: 31), 5: MR₆AK (SEQ ID No: 32), and 6: MR₈AK (SEQ ID No: 33).

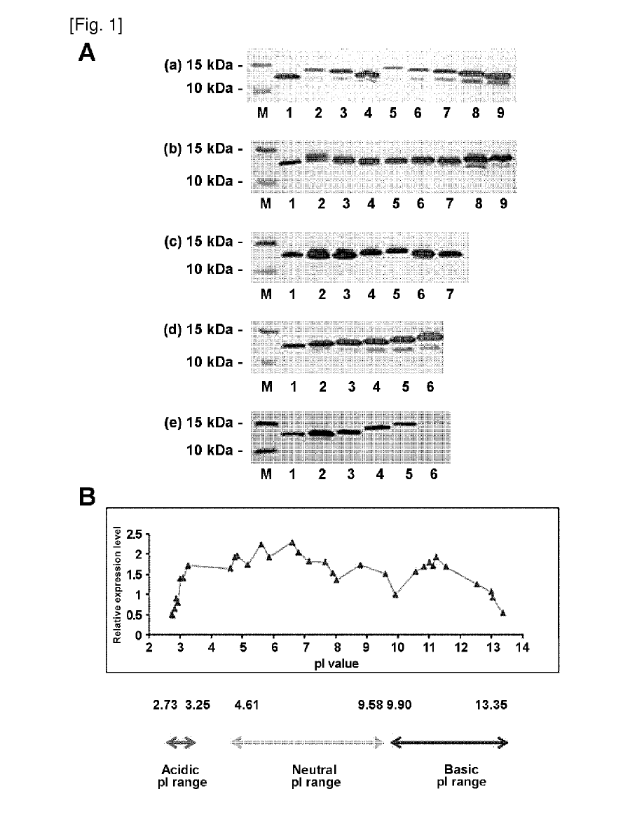

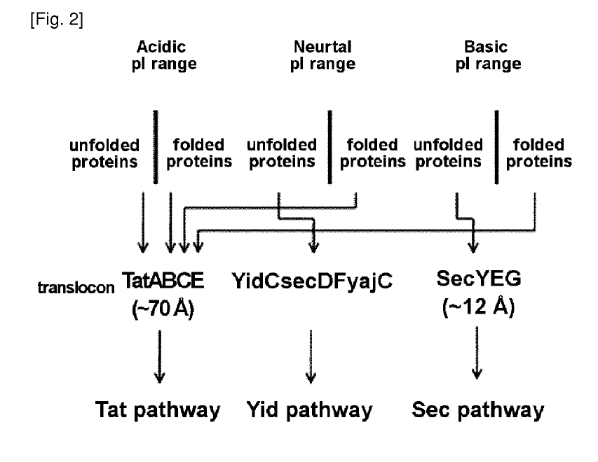

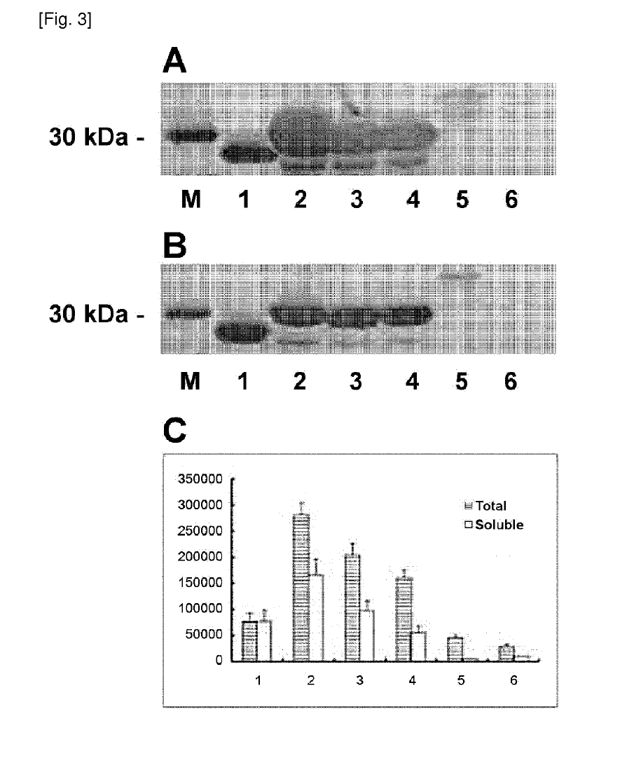

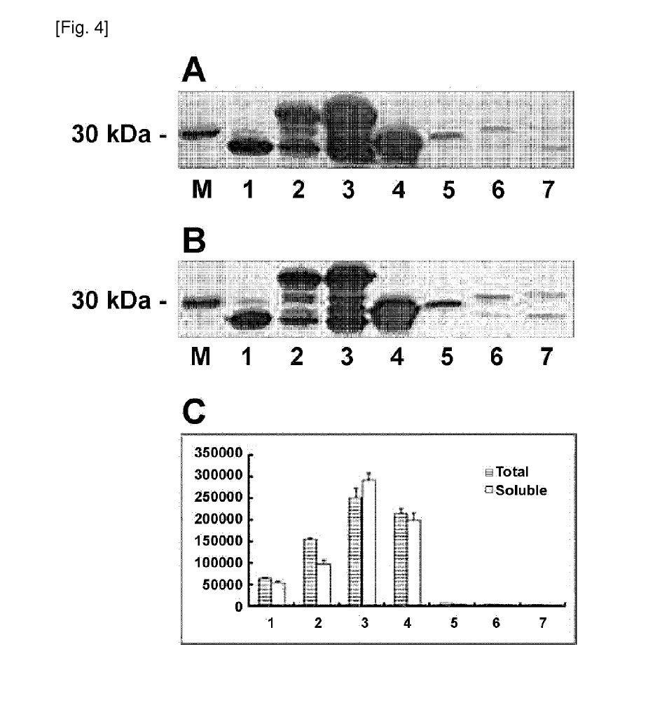

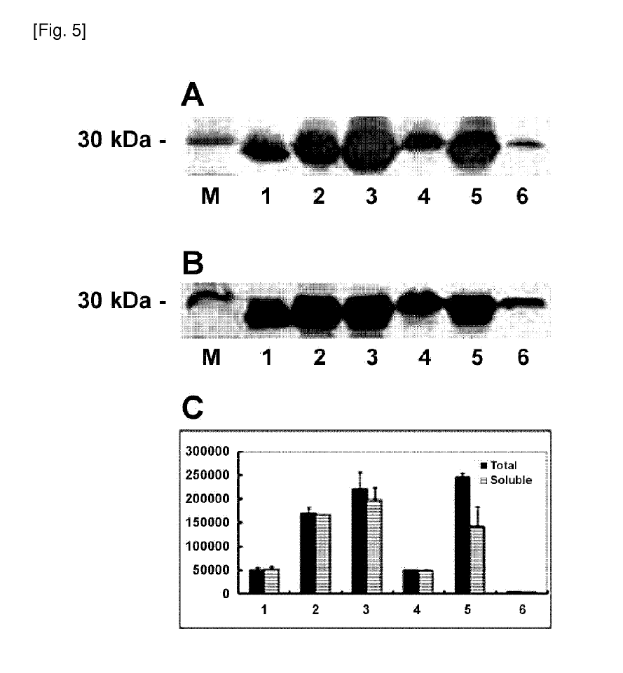

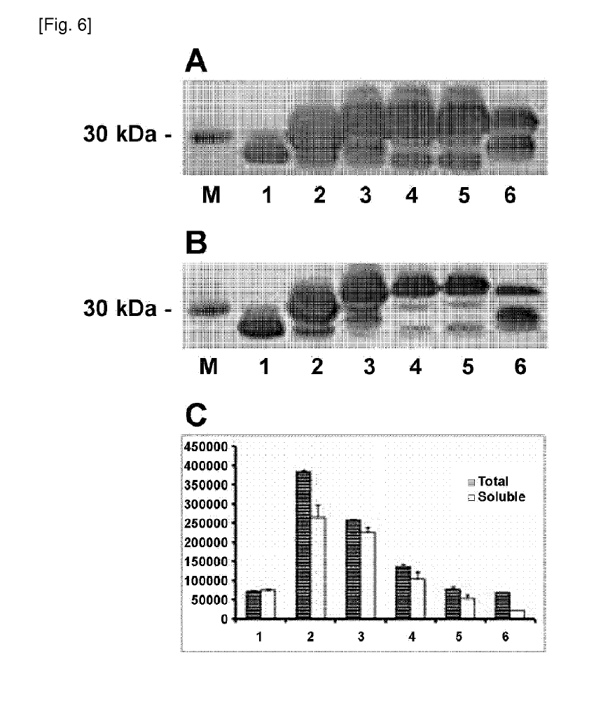

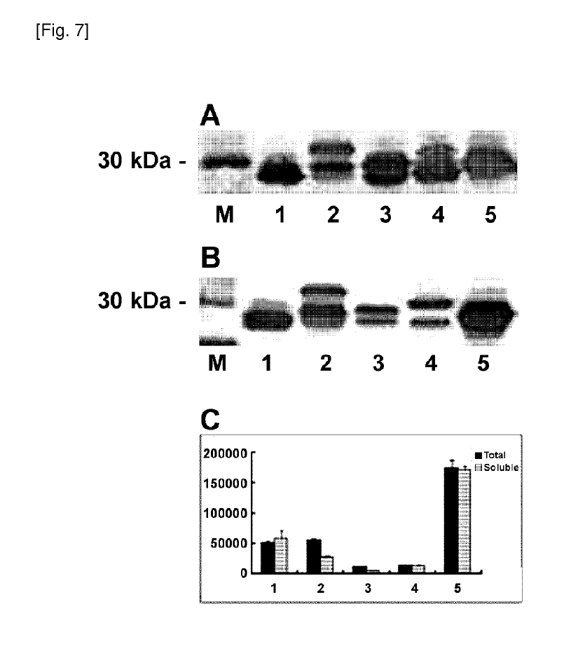

FIG. 1B is a graph showing soluble expression curve of rMefp1 at broad pI value range based on the result of Western blot analysis of FIG. 1A.

FIG. 2 is a schematic diagram showing type-II periplasmic secretion pathway at three specific pI ranges, acidic, neutral and basic, predicted from the soluble expression curve of FIG. 1B.

FIG. 3 is a series of photographs of Western blots of whole fraction (A) and soluble fraction (B) of clones transformed with expression vectors having gene constructions sequentially consisting of a polynucleotide encoding various variants of OmpASP₁₋₈ having modified pI value (Met-(X)(Y)-TAIAI(OmpASP₄₋₈)), 8 Arg and a polynucleotide encoding GFP, and a graph (C) showing the result of fluorescent assay of both the fractions:

| M: marker, | |
|---|---|
| (SEQ ID No: 115) | |
| lane 1: | GFP; |
| (SEQ ID No: 101) | |
| lane 2: | MEE-TAIAI-8Arg-GFP; |
| (SEQ ID No: 102) | |
| lane 3: | MAA-TAIAI-8Arg-GFP; |
| (SEQ ID No: 103) | |
| lane 4: | MAH-TAIAI-8Arg-GFP; |
| (SEQ ID No: 104) | |
| lane 5: | MKK-TAIAI-8Arg-GFP; |
| and | |
| (SEQ ID No: 105) | |
| lane 6: | MRR-TAIAI-8Arg-GFP. |

FIG. 4 is a series of photographs of Western blots of whole fraction (A) and soluble fraction (B) of clones transformed with expression vectors having gene constructions sequentially consisting of a polynucleotide encoding various leader peptides and a polynucleotide encoding GFP, wherein the leader peptides consist of homotype acidic or basic hydrophilic amino acids linked to methionine (Met), and a graph (C) showing the result of fluorescent assay of the two fractions:

| M: marker; | |
|---|---|
| (SEQ ID No: 115) | |
| lane 1: | GFP; |
| (SEQ ID No: 106) | |
| lane 2: | MDDDDDD; |
| (SEQ ID No: 107) | |
| lane 3: | MEEEEEE; |
| (SEQ ID No: 108) | |
| lane 4: | MKKKKKK; |
| (SEQ ID No: 109) | |
| lane 5: | MRRRRRR; |
| (SEQ ID No: 110) | |
| lane 6: | MRRRRRRRRR; |
| and | |
| (SEQ ID No: 111) | |
| lane 7: | MRRRRRRRRRRRR. |

FIG. 5 is a series of photographs of Western blots of whole fraction (A) and soluble fraction (B) of clones transformed with expression vectors having gene constructions sequentially consisting of a polynucleotide encoding various leader peptides and a polynucleotide encoding GFP, wherein the leader peptides consist of homotype and heterotype acidic or basic hydrophilic amino acids linked to methionine and wherein the polynucleotides encoding the leader peptides have various ΔG_{RNA}value, and a graph (C) showing the result of fluorescent assay of the two fractions:

| M: marker; | |
|---|---|
| (SEQ ID No: 115) | |
| lane 1: | GFP; |
| (SEQ ID No: 108) | |
| lane 2: | MKKKKKK(Lys^{AAA})₆; |
| (SEQ ID No: 112) | |
| lane 3: | MKKRKKR-I (Lys^{AAA}Lys^{AAA}Arg^{CGC})₂; |
| (SEQ ID No: 113) | |
| lane 4: | MKKRKKR-II (Lys^{AAG}Lys^{AAA}Arg^{CGC}); |
| (SEQ ID No: 114) | |
| lane 5: | MRRKRRK (Arg^{CGT}Arg^{CGC}Lys^{AAA})₂; |
| and | |
| (SEQ ID No: 109) | |
| lane 6: | MRRRRRR (Arg^{CGT}Arg^{CGC})₃. |

FIG. 6 is a series of photographs of Western blots of whole fraction (A) and soluble fraction (B) of clones transformed with expression vectors having a gene encoding modified GFP, wherein one or more amino acids among the 2^{nd} to 5^{th} amino acids of the GFP are substituted to glutamate, and a graph (C) showing the result of fluorescent assay of the two fractions:

| M: marker; | |
|---|---|
| (GFP₁₋₇, control, SEQ ID No: 115) | |
| lane 1: | MVSKGEE; |
| (GFP₁₋₇(V2E), SEQ ID No: 116) | |
| lane 2: | MESKGEE; |
| (GFP₁₋₇(V2E-S3E), SEQ ID No: 117) | |
| lane 3: | MEEKGEE; |
| (GFP₁₋₇(V2E-S3E-K4E), SEQ ID No: 118) | |
| lane 4: | MEEEGEE; |
| (GFP₁₋₇(V2E-S3E-K4E-G5E), SEQ ID No: 119) | |
| lane 5: | MEEEEEE; |
| and | |
| (SEQ ID No: 120) | |
| lane 6: | TorAss-GFP, control. |

FIG. 7 is a series of photographs of Western blots of whole fraction (A) and soluble fraction (B) of clones transformed with expression vectors having a gene construct sequentially consisting of a polynucleotide encoding a modified OmpA signal sequence whose N-terminal is substituted with a leader peptide, MKKKKKK which has basic pI and high hydrophilicity, and a graph (C) showing the result of fluorescent assay of the two fractions:

| M: marker; | |
|---|---|
| (SEQ ID No: 115) | |
| lane 1: | GFP, control; |
| (SEQ ID No: 120) | |
| lane 2: | TorAss-GFP, control, |
| (SEQ ID No: 121) | |
| lane 3: | OmpAss₁₋₃-OmpAss₄₋₂₃-GFP; |
| (SEQ ID No: 122) | |
| lane 4: | MKKKKKK-OmpAss₄₋₂₃-GFP; |
| and | |
| (SEQ ID No: 108) | |
| lane 5: | MKKKKKK-GFP. |

### BEST MODE FOR CARRYING OUT THE INVENTION

According to an aspect of the present invention, an expression vector for enhancing soluble expression and secretion of bulky folded active heterologous proteins having one or more inherent transmembrane-like domains or intramolecular disulfide bonds, comprising a gene construct consisting of: 1) a promoter; and, 2) a polynucleotide operably linked to the promoter, encoding a leader peptide having N-terminal whose pI value is 2.00 to 9.60 and whose hydrophilicity is 1.00 to 2.00 is provided.

The expression vector may consist of one or more replication origin; one or more selective marker; a gene construct for expression of a heterologous protein consisting sequentially of a promoter, a polynucleotide operably linked to the promoter, encoding a leader peptide having N-terminal whose pI value is 2.00 to 9.60 and whose hydrophilicity is 1.00 to 2.00; and optionally a multicloning site for inserting a polynucleotide encoding the heterologous protein operably. The expression vector may further comprise a transcription terminator operably linked to the gene construct, in order to enhance transcription efficiency. The expression vector may further comprise a polynucleotide corresponding to a protease recognition site operably linked to the gene construct. In addition, the expression vector may further comprise a polynucleotide encoding the heterologous protein operably linked to the polynucleotide encoding the leader peptide or the polynucleotide corresponding to a protease recognition site. Further, the expression vector may contain one or more enhancers if the vector is a eukaryotic vector.

According to an aspect of the present invention, a gene construct consisting of: 1) a promoter; and, 2) a polynucleotide operably linked to the promoter, which encodes a leader peptide having N-terminal whose pI value is 2.00 to 9.60 and whose hydrophilicity is 1.00 to 2.00 is provided.

According to an aspect of the present invention, a method for enhancing soluble expression and secretion of a bulky folded active heterologous protein having one or more inherent transmembrane-like domains or intramolecular disulfide bonds comprising:

Providing a polynucleotide encoding a leader peptide having N-terminal whose pI value is 2.00 to 9.60 and whose hydrophilicity is 1.00 to 2.00;

Constructing a gene construct consisting of the polynucleotide and a polynucleotide encoding the bulky folded active heterologous protein having one or more inherent transmembrane-like domains or intramolecular disulfide bonds;

Constructing a recombinant expression vector by operably inserting the gene construct into an expression vector;

Producing transformants by transforming host cells with the recombinant expression vector; and,

Selecting a transformant whose ability for expressing and secreting the bulky folded active heterologous protein is good among the transformants is provided.

According to an aspect of the present invention, a method for producing a bulky folded active heterologous protein having one or more inherent transmembrane-like domains or intramolecular disulfide bonds comprising:

Providing a polynucleotide encoding a leader peptide having N-terminal whose pI value is 2.00 to 9.60 and whose hydrophilicity is 1.00 to 2.00;

Constructing a gene construct encoding a fusion protein sequentially consisting of the leader peptide, a protease recognition site and the bulky folded active heterologous protein having one or more inherent transmembrane-like domains or intramolecular disulfide bonds;

Constructing a recombinant expression vector by operably inserting the gene construct into an expression vector;

Producing transformants by transforming host cells with the recombinant expression vector; and,

Culturing the transformants by inoculating culture media with the transformants;

Isolating the fusion protein; and

Isolating a native form of the bulky folded active heterologous protein after cleaving the protease recognition site with a protease is provided.

In the expression vector, the gene construct and the method, the promoter may be a viral promoter, a prokaryotic promoter or a eukaryotic promoter. The viral promoter may be cytomegalovirus (CMV) promoter, polioma virus promoter, fowl pox virus promoter, adenovirus promoter, bovine papilloma virus promoter, avian sarcoma virus promoter, retrovirus promoter, hepatitis B virus promoter, herpes simplex virus thymidine kinase promoter, simian virus 40 (SV40) promoter. The prokaryotic promoter may be T7 promoter, SP6 promoter, heat-shock protein (HSP) 70 promoter, -lactamase promoter, lac operon promoter, alkaline phosphatase promoter, trp operon promoter, or tac promoter. The eukaryotic promoter may be a yeast promoter, a plant promoter, or an animal promoter. The yeast promoter may be 3-phosphoglycerate kinase (PGK-3) promoter, enolase promoter, glyceraldehyde-3-phosphate dehydrogenase promoter, hexokinase promoter, pyruvate decarboxylase promoter, phosphofructokinase promoter, glucose-6-phosphate isomerase promoter, 3-phosphoglycerate mutase promoter, pyruvate kinase promoter, triosephosphate isomerase promoter, phosphoglucose isomerase promoter, glucokinase promoter, alcohol dehydrogenase 2 promoter, isocytochrome C promoter, acidic phosphatase promoter, Saccharomyces cerevisiae GAL1 promoter, Saccharomyces cerevisiae GAL7 promoter, Saccharomyces cerevisiae GAL10 promoter, or Pichia pastoris AOX1 promoter. The animal promoter may be heat-shock protein promoter, proactin promoter or immunoglobulin promoter.

However, any promoters can be used if they normally express heterologous proteins in host cells.

The pI value may be 2.56 to 7.65 or the pI value may be 2.56 to 5.60. Alternatively, the pI value may be 2.73 to 3.25.

The hydrophilicity may be between 1.16 and 1.82. In the meantime, the hydrophilicity may be a value according to Hopp-Woods (Hopp and Woods, Proc. Natl. Acad. Sci. USA, 78: 3824-3828, 1981).

The leader peptide may be a variant of a signal peptide fragment, or may have additionally 1 to 30 hydrophilic amino acids linked thereto. The signal peptide fragment may be a peptide in which the 2^{nd} and/or the 3^{rd} amino acid of N-terminal of the variant is substituted with aspartate (Asp) or glutamate (Glu). The hydrophilic amino acid may be Asp, Glu, glutamine (Gln), asparagine (Asn), threonine (Thr), serine (Ser), arginine (Arg) or lysine (Lys). The variant may be a full-length of the signal peptide or may consist of 2 to 20 amino acids. The variant may consist of 2 to 12 amino acids or 3 to 10 amino acids. The leader peptide may have amino acid sequence of SEQ ID Nos: 101 to 103.

The signal peptide may be a viral signal sequence, a prokaryotic signal sequence or a eukaryotic signal sequence. More particularly, the signal sequence may be OmpA signal sequence, CT-B (cholera toxin subunit B) signal sequence, LTIIb-B (E. coli heat-labile enterotoxin B subunit) signal sequence, BAP (bacterial alkaline phosphatase) signal sequence (Izard and Kendall, Mol. Microbiol. 13:765-773, 1994), Yeast carboxypeptidase Y signal sequence (Blachly-Dyson and Stevens, J. Cell. Biol. 104: 1183-1191, 1987), Kluyveromyces lactis killer toxin gamma subunit signal sequence (Stark and Boyd, EMBO J. 5(8): 1995-2002, 1986), bovine growth hormone signal sequence (Lewin, B. (Ed), GENES V, p290. Oxford University Press, 1994), influenza neuraminidase signal-anchor (Lewin B. (Ed), GENES V, p297. Oxford University Press, 1994), Translocon-associated protein subunit alpha, TRAP—(Prehn et al., Eur. J. Biochem. 188(2): 439-445, 1990) signal sequence, Twin-arginine translocation (Tat) signal sequence (Robinson, Biol. Chem. 381(2): 89-93, 2000).

Alternatively, the leader peptide may be a synthetic peptide having 1 to 30 hydrophilic amino acids linked to the first amino acid, methionine. Alternatively, the synthetic peptide may consist of 3 to 16 amino acids linked to carboxy-terminal of Met, wherein at least 60% of the amino acids are hydrophilic. The hydrophilic amino acids may be homotypic or heterotypic. The hydrophilic amino acids may be selected from a group consisting of Asp, Glu, Gln, Asn, Thr, Ser, Arg, and Lys. In a more particular example, the leader peptide may have an amino acid sequence selected from a group consisting of SEQ ID Nos: 1-22, 106, 107, 116, 117 and 118.

The length of the leader peptide may be 1 to 30 amino acids, 2 to 20 amino acids, 4 to 10 amino acids, or 6 to 8 amino acids.

The protease recognition site may be Xa factor recognition site, enterokinase recognition site, Genenase I recognition site or Furin recognition site or a combination thereof may be used. If a protease to be used is Xa factor, the protease recognition site may be Ile-Glu-Gly-Arg. In addition, between the polynucleotide encoding the leader peptide and the protease recognition site, one to three neutral amino acids such as neutral nonpolar amino acids selected from a group consisting of Gln, Ala, Val, Leu, Ile, Phe, Trp, Met, Cys and Pro or neutral polar amino acids selected from a group consisting of Ser, Thr, Tyr, Asn and Gln may be additionally inserted.

The bulky folded protein may have one or more transmembrane domains, transmembrane-like domains, amphipathic domains or intramolecular disulfide bonds. In an example, the bulky folded protein may be green fluorescent protein (GFP). A heterologous protein having the transmembrane domains, transmembrane-like domains, or amphipathic domains is assumed to be secreted hardly into the periplasm because a region having positive charge may attach to lipid bilayer of membrane and the transmembrane-like domain may play a role as an anchor. In order to secret these unsecretable proteins into the periplasm, the expression vector of the present invention is very effective.

The expression vector is suitable to produce heterologous proteins having transmembrane domain, transmembrane-like domain or amphipathic domain in soluble form. This is assumed that the secretion of expressed heterologous protein is enhanced because the directional force and the effect of high hydrophilicity of a leader peptide is bigger than the force which the domains attach to the lipid bilayer, when the hydrophilicity of the leader peptide of the present invention is bigger than that of the transmembrane domain existing in the heterologous protein.

Further, when the expressed heterologous protein is secreted into the periplasm, the heterologous protein has different secretional pathways according to pI value of N-terminal of the heterologous protein. Particularly, when N-terminal of a heterologous protein has acidic pI value, the heterologous protein is secreted through Tat pathway E. coli type-II periplasmic secretion pathway. Although a leader peptide is one which is secreted through other pathways, a bulky folded active heterologous protein linked thereto is secreted through the Tat pathway. Therefore, if a heterologous protein is a bulky protein whose folded form is active, we can enhance secretional efficiency of the heterologous protein by adjusting pI value of the leader peptide to acidic range and selecting Tat pathway thereby (See FIG. 2).

According to an aspect of the present invention, an expression vector for enhancing soluble expression and secretion of bulky folded active heterologous proteins having one or more inherent transmembrane-like domains or intramolecular disulfide bonds, comprising a gene construct consisting of: 1) a promoter; and, 2) a polynucleotide operably linked to the promoter, encoding a leader peptide having N-terminal whose pI value is 9.90 to 13.35 and whose hydrophilicity is 1.00 to 2.50, wherein the polynucleotide has ΔG_{RNA} value of more than −10.00 is provided. The expression vector may further comprise a transcription terminator operably linked to the gene construct for enhancing transcription efficiency.

The expression vector may consist of one or more replication origin; one or more selective marker; a gene construct for expression of a heterologous protein consisting sequentially of a promoter, a polynucleotide operably linked to the promoter, encoding a leader peptide having N-terminal whose pI value is 9.90 to 13.35 and whose hydrophilicity is 1.00 to 2.50, wherein the polynucleotide has ΔG_{RNA} value of more than −10.00; and optionally a multicloning site for inserting a polynucleotide encoding the heterologous protein operably. The expression vector may further comprise a polynucleotide corresponding protease recognition site operably linked to the gene construct. In addition, the expression vector may further comprise a polynucleotide encoding the heterologous protein operably linked to the polynucleotide encoding the leader peptide or the polynucleotide corresponding to a protease recognition site. Further, the expression vector may contain one or more enhancers if the vector is a eukaryotic vector.

According to an aspect of the present invention, a gene construct consisting of: 1) a promoter; and, 2) a polynucleotide operably linked to the promoter, encoding a leader peptide having N-terminal whose pI value is 9.90 to 13.35 and whose hydrophilicity is 1.00 to 2.50, wherein the polynucleotide has ΔG_{RNA} value of more than −10.00 is provided.

According to another aspect of the present invention, a method for enhancing soluble expression and secretion of a bulky folded active heterologous protein having one or more inherent transmembrane-like domains or intramolecular disulfide bonds, the method comprising:

Providing a polynucleotide encoding a leader peptide having N-terminal whose pI value is 9.90 to 13.35 and whose hydrophilicity is 1.00 to 2.50, wherein the polynucleotide has ΔG_{RNA}value of more than −10.00;

Constructing a gene construct consisting of the polynucleotide and a polynucleotide encoding the bulky folded active heterologous protein having one or more inherent transmembrane-like domains or intramolecular disulfide bonds, wherein the bulky folded active heterologous protein moves into the periplasm as a folded form and has biological activity in the periplasm;

Constructing a recombinant expression vector by operably inserting the gene construct into an expression vector;

Producing transformants by transforming host cells with the recombinant expression vector; and,

Selecting a transformant whose ability for expressing and secreting the bulky folded active heterologous protein is good among the transformants is provided.

In the expression vector, the gene construct and the method, the promoter may be a viral promoter, a prokaryotic promoter or a eukaryotic promoter. The viral promoter may be cytomegalovirus (CMV) promoter, polioma virus promoter, fowl pox virus promoter, adenovirus promoter, bovine papilloma virus promoter, avian sarcoma virus promoter, retrovirus promoter, hepatitis B virus promoter, herpes simplex virus thymidine kinase promoter, or simian virus 40 (SV40) promoter. The prokaryotic promoter may be T7 promoter, SP6 promoter, heat-shock protein (HSP) 70 promoter, -lactamase promoter, lac operon promoter, alkaline phosphatase promoter, trp operon promoter, or tac promoter. The eukaryotic promoter may be a yeast promoter, a plant promoter, or an animal promoter. The yeast promoter may be 3-phosphoglycerate kinase (PGK-3) promoter, enolase promoter, glyceraldehyde-3-phosphate dehydrogenase promoter, hexokinase promoter, pyruvate decarboxylase promoter, phosphofructokinase promoter, glucose-6-phosphate isomerase promoter, 3-phosphoglycerate mutase promoter, pyruvate kinase promoter, triosephosphate isomerase promoter, phosphoglucose isomerase promoter, glucokinase promoter, alcohol dehydrogenase 2 promoter, isocytochrome C promoter, acidic phosphatase promoter, Saccharomyces cerevisiae GAL1 promoter, Saccharomyces cerevisiae GALT promoter, Saccharomyces cerevisiae GAL10 promoter, or Pichia pastoris AOX1 promoter. The animal promoter may be heat-shock protein promoter, proactin promoter or immunoglobulin promoter.

However, any promoters can be used if they normally express heterologous proteins in host cells.

The pI value may be 10 to 13.2 or 11 to 13.

The hydrophilicity may be adjusted between 1 and 2.5. In the meantime, the hydrophilicity may be a value according to Hopp-Woods (Hopp and Woods, Proc. Natl. Acad. Sci. USA, 78: 3824-3828, 1981).

The G_{RNA} value may be adjusted between −7.6 and 1.6, −5 to 1.0 or −3 to 0.6.

The leader peptide may be a variant of a signal peptide fragment, or may have additionally 1 to 30 hydrophilic amino acids linked thereto. The signal peptide fragment may be a peptide in which the 2^{nd} and/or the 3^{rd} amino acid of N-terminal of the variant is substituted with aspartate (Asp) or glutamate (Glu). The hydrophilic amino acid may be Asp, Glu, glutamine (Gln), asparagine (Asn), threonine (Thr), serine (Ser), arginine (Arg) or lysine (Lys). The variant may be a full-length of the signal peptide or may consist of 2 to 20 amino acids. The length of the leader peptide may be 1 to 30 amino acids, 2 to 20 amino acids, 4 to 10 amino acids, or 6 to 8 amino acids. In a more particular example, the leader peptide has amino acid sequence of SEQ ID Nos: 104 or 105.

The signal peptide may be a viral signal sequence, a prokaryotic signal sequence or a eukaryotic signal sequence. More particularly, the signal sequence may be OmpA signal sequence, CT-B (cholera toxin subunit B) signal sequence, LTIIb-B (E. coli heat-labile enterotoxin B subunit) signal sequence, BAP (bacterial alkaline phosphatase) signal sequence (Izard and Kendall, Mol. Microbiol. 13:765-773, 1994), Yeast carboxypeptidase Y signal sequence (Blachly-Dyson and Stevens, J. Cell. Biol. 104: 1183-1191, 1987), Kluyveromyces lactis killer toxin gamma subunit signal sequence (Stark and Boyd, EMBO J. 5(8): 1995-2002, 1986), bovine growth hormone signal sequence (Lewin, B. (Ed), GENES V, p290. Oxford University Press, 1994), influenza neuraminidase signal-anchor (Lewin B. (Ed), GENES V, p297. Oxford University Press, 1994), Translocon-associated protein subunit alpha, TRAP- (Prehn et al., Eur. J. Biochem. 188(2): 439-445, 1990) signal sequence, Twin-arginine translocation (Tat) signal sequence (Robinson, Biol. Chem. 381(2): 89-93, 2000).

Alternatively, the leader peptide may be a synthetic peptide having 1 to 30 hydrophilic amino acids linked to the first amino acid, methionine. Alternatively, the synthetic peptide may consist of 3 to 16 amino acids linked to carboxy-terminal of Met, wherein at least 60% of the amino acids are hydrophilic. The hydrophilic amino acids may be homotypic or heterotypic. The hydrophilic amino acids may be selected from a group consisting of Asp, Glu, Gln, Asn, Thr, Ser, Arg, and Lys. In a more particular example, the leader peptide may have amino acid sequence of SEQ ID Nos: 24-33, 108-114.

Further, when the N-terminal of a heterologous protein has basic pI value and moves to the periplasm as unfolded and then is folded in periplasm, the heterologous protein is secreted through Sec pathway E. coli type-II periplasmic secretion pathway. Therefore, if a heterologous protein is a protein which moves to the periplasm as unfolded and then is folded in the periplasm, we can enhance secretional efficiency of the heterologous protein by adjusting pI value of the leader peptide to basic range and selecting Sec pathway thereby (See FIG. 2).

Hereinafter, terms and phrases used in the present document are described.

The phrase “heterologous protein” refers to a protein to be produced by genetic recombination technique, more particularly it is a protein expressed in host cells transformed with an expression vector having a polynucleotide encoding the protein.

The phrase “fusion protein” refers to a protein in which another polypeptide is linked or additional amino acid sequence is added to an N- or C-terminal of an original heterologous protein.

The term “folding” refers to a process that a primary polypeptide chain gets unique tertiary structure exhibiting its function via structural deformation.

The phrase “folded active protein” refers to a protein forming tertiary structure in order to possess the inherent activity in the cytosol after the transcription and the translation of mRNA or before the secretion into the periplasm.

The phrases “signal peptide (SP)” and “signal sequence (ss)” which may be used interchangeably other in the art refer to a peptide helping a heterologous protein expressed from viruses, prokaryotes or eukaryotes pass cellular membrane in order to secrete the heterologous protein into the periplasm or outside the cell or into the target organ. Although it seemed that the “signal sequence” does not designate a molecule but sequence information, the “signal sequence” is recognized to designate a polypeptide molecule. Generally the signal sequence consists of positively charge N-region, central characteristic hydrophobic region, and c-region with a cleavage site. The phrase “signal peptide fragment” used herein refers to a whole region or a part of positively charged N-region, central characteristic hydrophobic region, and c-region with cleavage site. In addition, the signal sequence includes Sec signal sequence and Tat signal sequence which have these three parts.

The term “hydrophilicity” refers to extent capable of forming hydrogen bond with water molecules. Unless otherwise defined, the hydrophilicity value is calculated according to Hopp-Woods scale using DNASIS™ (Hitachi, Japan) software (window size: 6 and threshold: 0.00). The term “hy” is an abbreviation of the term “hydrophilicity”. When the hydrophilicity value of a peptide is positive the peptide is hydrophilic and the hydrophilicity value is negative the peptide is hydrophobic.

The phrase “leader peptide” or “leader sequence” refers to an additional amino sequence added to N-terminal of a heterologous protein.

The phrase “N-terminal of a leader peptide” refers to 1 to 10 amino acids located in the amino terminal of the leader peptide.

The term “fragment” refers to a peptide or a polynucleotide having minimum length but maintaining the function of full-length peptide or full-length polynucleotide. Unless otherwise defined, the fragment neither includes the full-length peptide nor the full-length polynucleotide. For example, “signal peptide fragment” used in the present document refers to a truncated signal peptide with the deletion of C-terminal cleavage region or central hydrophobic region and the C-terminal cleavage region, which plays a role as a signal sequence and does not include a full-length signal sequence.

The term “polynucleotide” refers to a polymer molecule in which two or more nucleotide molecules are linked one another through phosphodiester bond and DNA and RNA are included therein.

The phrase “N-terminal region of a signal peptide” refers to a conservative region found common signal sequences which 1 to 10 amino acid of amino terminal of a signal peptide.

The phrase “variant of signal peptide fragment” refers to a peptide whose one or more amino acids at any position except the 1^{st} methionine are substitute with other amino acids.

The phrase “protease recognition site” means an amino acid sequence which a protease recognizes and cleaves.

The phrase “transmembrane domain” refers to a domain having hydrophilic region and hydrophobic region in turn, and means an internal region of a protein having a similar structure with amphipathic domain. Therefore, it is used as the same meaning as “transmembrane-like domain”.

The phrase “transmembrane-like domain” refers to a region predicted to have similar structure as the transmembrane domain of a membrane protein when analyzing amino acid sequence of a polypeptide (Brasseur et al., Biochim. Biophys.Acta 1029(2): 267-273, 1990). Usually it can be easily predicted with various computer softwares which predict transmembrane domains. In particular examples of the computer softwares, there are TMpred, HMMTOP, TBBpred, DAS-TMfilter (www.enzim.hu/DAS/DAS.html), etc. The “transmembrane-like domain” includes a “transmembrane domain” which is revealed to pass through membranes indeed.

The phrase “expression vector” refers to a linear or a circular DNA molecule comprising all cis-acting elements for expressing a heterologous protein such as a promoter, a terminator or an enhancer. Conventional expression vectors have a multi cloning site with various restriction sites for cloning a polynucleotide encoding the heterologous protein. However, the expression vector used in the present document includes one including the polynucleotide encoding the heterologous. In addition, the expression vector may further contain one or more replication origins, one or more selective markers, a polyadenylation signal, etc. The expression vector contains elements originated from a plasmid and/or a virus generally.

The phrase “operably linked to” or “operably inserted to” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “ΔG_{RNA} value” refers to Gibson free energy level which an RNA has in aqueous solution at particular temperature. However when ΔG_{RNA} value is low, it is expressed that the Gibson free energy is high. Thus lower the value is, more stable the secondary structure is maintained. For example, an RNA whose ΔG_{RNA} value is −10 has bigger Gibson free energy than one has ΔG_{RNA} value of −2 and thus the former has more stable secondary structure than the letter.

### MODE FOR THE INVENTION

Hereinafter, the present invention is described below with particular examples.

However, the following examples serve to illustrate the present invention and are not intended to limit its scope in any way.

### Example 1

### Analysis of Soluble Expression of a Protein According to pI Value of N-Terminal of a Leader Peptide

The present inventors designated a DNA repeat sequence consisting of 7 repeats of a polynucleotide encoding Mefp1 having the amino acid sequence Ala Lys Pro Ser Tyr Pro Pro Thr Tyr Lys (SEQ ID No: 153) as 7mefp1 in previous work (Korean Patent No: 981356) and analyzed the extent of soluble expression of heterologous proteins encoded by the DNA repeat sequence operably linked to polynucleotides encoding various N-terminal leader peptides having broad range of pI value (2.73 to 13.35) based on another work (Korean Patent Gazette No: 2009-0055475, See Tables 1 and 2).

<1-1> Construction of Expression Vectors Having Gene Constructs Comprising Polynucleotides Encoding Recombinant 7Mefp1 Having Broad Range of pI Value

The present inventors constructed pET-22b(+)(ompASP₁(Met)-7mefp1*) which is a N-terminal fused plasmid by introducing OmpASP₁(Met) and 7mefp1 into pET-22b(+) vector using the method described in Korean Patent Gazette No: 2009-0055457 and then constructed 33 pET-22b(+) clones which have polynucleotides encoding a fusion protein consisting of various leader peptide (SEQ ID Nos: 1-33) with broad range of pI value (2.73 to 13.35) and 7Mefp1 whereby performing PCR reactions using forward primers having nucleotide sequence of SEQ ID Nos: 34-66), a reverse primer having nucleotide sequence of SEQ ID No: 67 and pET-22b(+)(ompASP₁(Met)-7mefp1*) as a template (Table 1).

TABLE 1
Relative soluble expression level of rMefp1 according to various pI value
of N-terminal of leader peptides

| | a.a | | | | |
|---|---|---|---|---|---|
| | sequence | | | | |
| | of N- | | | | Relative |
| SEQ | terminal | | SEQ | | soluble |
| ID | of leader | pI | ID | Forward primers used for | expres- |
| Nos | peptide | value | Nos | designing leader seuqences | sion |
| 1* | MDDDDDAA | 2.73 | 34 | CAT ATG GAC GAT GAC GAT GAC GCT GCA CCG TCT TAT CCG CCA | 0.50 |
| 2* | MDDDAA | 2.87 | 35 | CAT ATG GAC GAT GAG GCT GCA CCG TCT TAT CCG CCA ACC TA [symbol] | 0.91 |
| 3 | MDA | 3.00 | 36 | CAT ATG GAC GCT CCG TCT TAT CCG CCA ACC TAC | 1.40 |
| 4 | MEEEEEEEE | 2.75 | 37 | CAT ATG GAA GAG GAA GAG GAA GAG GAA GAG CCG TCT TAT CCG | 0.49 |
| 5 | MEEEEEE | 2.82 | 38 | CAT ATG GAA GAG GAA GAG GAA GAG CCG TCT TAT CCG CCA AC [symbol] | 0.65 |
| 6 | MEEEE | 2.92 | 39 | CAT ATG GAA GAG GAA GAG CCG TCT TAT CCG CCA ACC TAC | 0.79 |
| 7* | MFE | 3.09 | 40 | CAT ATG GAA GAG CCG TCT TAT CCG CCA ACC TAC | 1.42 |
| 8* | MAE | 3.25 | 41 | CAT ATG GCT GAA CCG TCT TAT CCG CCA ACC TAC | 1.72 |
| 9 | MCCCCCC | 4.61 | 42 | CAT ATG TGC TGT TGC TGT TGC TGT CCG TCT TAT CCG CCA AC [symbol] | 1.65 |
| | | | | TAC | |
| 10 | MCCC | 4.75 | 43 | CAT ATG TGC TGT TGC CCG TCT TAT CCG CCA ACC TAC | 1.93 |
| 11 | MAC | 4.83 | 44 | CAT ATG GCT TGC CCG TCT TAT CCG CCA ACC TAC | 1.96 |
| 12 | MAY | 5.16 | 45 | CAT ATG GCT TAC CCG TCT TAT CCG CCA ACC TAC | 1.74 |
| 13* | MAA | 5.60 | 46 | CAT ATG GCT GCA CCG TCT TAT CCG CCA ACC TAC | 2.25 |
| 14 | MGG | 5.85 | 47 | CAT ATG GGT GGT CCG TCT TAT CCG CCA ACC TAC | 1.93 |
| 15 | MAKD | 6.59 | 48 | CAT ATG GCT AAA GAC CCG TCT TAT CCG CCA ACC TAC | 2.30 |
| 16 | MAKE | 6.79 | 49 | CAT ATG GCT AAA GAA CCG TCT TAT CCG CCA ACC TAC | 2.05 |
| 17* | MCH | 7.13 | 50 | CAT ATG TGC CAC CCG TCT TAT CCG CCA ACC TAC | 1.83 |
| 18* | MAH | 7.65 | 51 | CAT ATG GCT CAC CCG TCT TAT CCG CCA ACC TAC | 1.81 |
| 19 | MAHHH | 7.89 | 52 | CAT ATG GCT CAC CAT CAC CCG TCT TAT CCG CCA ACC TAC | 1.54 |
| 20 | MAHHHHH | 8.01 | 53 | CAT ATG GCT CAC CAT CAC CAT CAC CCG TCT TAT CCG CCA AC [symbol] | 1.37 |
| 21 | MAKC | 8.78 | 54 | CAT ATG GCT AAA TGC CCG TCT TAT CCG CCA ACC TAC | 1.73 |
| 22 | MKY | 9.58 | 55 | CAT ATG AAA TAC CCG TCT TAT CCG CCA ACC TAC | 1.51 |
| 23* | MAK | 9.90 | 56 | CAT ATG GCT AAG CCG TCT TAT CCG CCA ACC TAC | 1.00 |
| | (control) | | | | |
| 24* | MKAK | 10.55 | 57 | CAT ATG AAA GCT AAG CCG TCT TAT CCG CCA ACC TAC | 1.57 |
| 25 | MKKAK | 10.82 | 58 | CAT ATG AAA AAA GCT AAG CCG TCT TAT CCG CCA ACC TAC | 1.69 |
| 26* | MKKKAK | 10.99 | 59 | CAT ATG AAA AAA AAA GCT AAG CCG TCT TAT CCG CCA ACC TA [symbol] | 1.80 |
| 27* | MKKKKAK | 11.11 | 60 | CAT ATG AAA AAA AAA AAA GCT AAG CCG TCT TAT CCG CCA AC [symbol] | 1.72 |
| | | | | TAC | |
| 28* | MKKKKKAK | 11.21 | 61 | CAT ATG AAA AAA AAA AAA AAA GCT AAG CCG TCT TAT CCG CCA | 1.93 |
| 29 | MRAK | 11.52 | 62 | CAT ATG AGA GCT AAG CCG TCT TAT CCG CCA ACC TAC | 1.69 |
| 30* | MRRAK | 12.51 | 63 | CAT ATG CGT CGC GCT AAG CCG TCT TAT CCG CCA ACC | 1.26 |
| 31* | MRRRRAK | 12.98 | 64 | CAT ATG CGT CGC CGT CGC GCT AAG CCG TCT TAT CCG CCA AC [symbol] | 1.07 |
| 32* | MRRRRRRAK | 13.20 | 65 | CAT ATG CGT CGC CGT CGC CGT CGC GCT AAG CCG TCT TAT CCG | 0.93 |
| 33* | MRRRRRRRRAK | 13.35 | 66 | CAT ATG CGT CGC CGT CGC CGT CGC CGT CGC GCT AAG CCG TCT | 0.55 |
| | | | | TAT CCG CCA ACC | |

| | | |
|---|---|---|
| Reverse primer | 67 | CTC GAG GTC GAC AAG CTT ACG |

CAT: Extended for preserving Nde I site.
Bold characters refer to polynucleotides encoding signal peptide variant effecting pI value.
Normal characters refer to polynucleotide encoding the 3^{rd} to the 8^{th} amino acid of Mefp1.
*Amino acid sequences of N-terminals of leader peptides and nucleotide sequence of forward primers corresponding to the amino acid sequences which are reported in Korean Patent Gazette No: 2009-0055457. [symbol]
indicates data missing or illegible when filed

<1-2> Analysis of the Extent of Soluble Expression of Recombinant Proteins Using 7Mefp1 Clones

E. coli BL21(DE3) was transformed with the expression vectors constructed above using a conventional method and the transformants were cultured in LB media (tryptone 20 g/L, yeast extract 5 g/L, NaCl0.5 g/L, KCl 1.86 mg/L) with 100 μg/L ampicillin overnight at 30 C and then the culture was diluted 100 times with LB media and cultured until OD₆₀₀ is 0.6. And then, 1 mM IPTG was added for induction and was further cultured for 3 hr. One ml of the culture was centrifuged at 4,000 g for 30 min at 4 C and pellet was suspended with 100 to 200 μl of PBS. The suspension was sonicated with 152-s cycle pulses (at 30% power output) in order to isolate proteins and then the sonicated solution was centrifuged at 16,000 rpm for 30 min at 4 C. Supernatant was taken as a soluble protein fraction. The protein fractions were quantified using Bradford method (Bradford, Anal. Biochem.,72: 248-254, 1976). And then, 20 μg of proteins per well were loaded on 15% SDS-PAGE gel and SDS-PAGE analyses were performed according to Laemmli (Nature, 227: 680-685, 1970). The gels were stained with Coomassie Brilliant Blue stain (Sigma, USA). In the meantime the gels after SDS-PAGE analyses were transferred to Hybond-P™ membrane; GE, USA. Since the expression vectors produce rMefp1 as a fusion protein linked to His tag, the extent of expression of the recombinant protein was quantified using anti-His tag antibody as a primary antibody and alkaline phosphatase-conjugated anti-mouse antibody was used as a secondary antibody. Finally the rMefp1 was detected with a chromogenic Western blotting kit (Invitrogen, USA) according to manufacturer's instruction (FIG. 1A). The band density of the recombinant proteins obtained by the above method was quantified with densitometer analyzing method using image analysis software (Quantity One 1-D image analysis software, Bio-Rad, USA). Soluble expression level was averaged with the result of the above Western blot analysis (FIG. 1A), and the extent of soluble expression of rMefp1 fusion protein having a leader peptide MAK (pI 9.90, SEQ ID No: 23) was used a control and designated as 1.00.

As a result, the present inventors acknowledged that there are three different soluble expression curves showing different features in acidic (pI 2.73-3.25), neutral (pI 4.61-9.58) and basic (pI 9.90-13.35) pI range, respectively (FIG. 1B). The acidic, neutral and basic pI ranges in soluble expression curve of rMefp1 of FIG. 1B were illustrated in red, yellow and blue lines, respectively.

Therefore, the present inventors hypothesized that recombinant proteins are secreted through 3 different inner membrane channels according to pI value of a leader peptide.

In addition, after analyzing soluble expression of rMefp1, in pI value of 3.00, 3.09 and 3.25 among acidic pI values higher expression level than control was observed, in all neutral pI value much higher expression level than control was observed, and in pI value of 10.55, 10.82, 10.99, 11.11, 11.21 and 11.52 among basic pI values much higher expression level than control was observed. Thus, it is acknowledged that using a leader peptide having basic pI value is beneficial for inducing soluble expression of a heterologous protein without transmembrane-like domain.

Further, after analyzing the characteristic of soluble expression of rMefp1, decrease of soluble expression level when using MD₅AA and ME₈ leader peptide whose pI value is acidic and having increased hydrophilic amino acids and MR₈AK whose pI value is basic was observed. From the result, we can hypothesize that soluble expression of a heterologous protein without transmembrane-like domain is related to pI value rather than increment of hydrophilicity, unlike soluble expression of Olive flounder hepcidin I was increased by using leader peptides including poly Lys and Arg (Korean Patent No: 981356) or poly Lys and Arg and poly Glu (Korean Patent Gazette No: 2009-0055457).

Soluble expression level was averaged with the result of the above Western blot analysis (FIG. 1A), and the extent of soluble expression of rMefp1 fusion protein having a leader peptide MAK (pI 9.90, SEQ ID No: 23) was used a control and designated as 1.00.

### Example 2

### Prediction of Protein Secretion According to pI Value and Hydrophilicity of N-Terminals of Leader Peptides

Although E. coli type-II periplasmic secretion pathway (Mergulhao et al., Biotechnol. Adv. 23: 177-202, 2005) is classified roughly as Sec pathway, SRP pathway and Tat pathway; the present inventors think that the classification is not perfect because the E. coli type-II periplasmic secretion pathway which is known as a pathway related to soluble expression of proteins is very complex. Thus, the present inventors analyzed the E. coli type-II periplasmic secretion pathway in a new classification, the pI value of N-terminal of a signal sequence as shown in Tables 2 and 3, based on our previous reports (Korean Patent Gazette No: 2009-0055457 and Lee et al., Mol. Cells 26: 34-40, 2008) which disclose that N-terminal fragment of a signal peptide with specific pI value can substitute for whole length of the signal sequence. The pI values of signal sequences were analyzed using computer software DNASIS™ (Hitachi, Japan).

TABLE 2
Amino acid sequences, pI value of N-terminal
and predicted pI curve of representative Sec
signal sequences

| | | | | Pre- |
|---|---|---|---|---|
| SEQ | Signal | | | dicted |
| ID | se- | | pI | pI |
| Nos | quences | Amino acid sequences | value | curve |
| 68 | PhoA | MKQSTIALALLPLLFTPVTKA | 9.90 | Basic |
| 69 | OmpA | MKKTAIAIAVALAGFATVAQA | 10.55 | Basic |
| 70 | StII | MKKNIAFLLASMFVFSIATNAYA | 10.55 | Basic |
| 71 | PhoE | MKKSTLALVVMGIVASASVQA | 10.55 | Basic |
| 72 | MalE | MKIKTGARILALSALTTMMFSASALA | 10.55 | Basic |
| 73 | OmpC | MKVKVLSLLVPALLVAGAANA | 10.55 | Basic |
| 74 | Lpp | MKATKLVLGAVILGSTLLAG | 10.55 | Basic |
| 75 | LTB | MNKVKCYVLFTALLSSLYAIIG | 10.55 | Basic |
| 76 | OmpF | MMKRNILAVIVPALLVAGTANA | 11.52 | Basic |
| 77 | LamB | MMITLRKLPLAVAVAAGVMSAQAMA | 11.52 | Basic |
| 78 | OmpT | MRAKLLGIVLTTPIAISSFA | 11.52 | Basic |

Signal sequences and N-domains thereof were adopted as referenced (Choi and Lee, Appl. Microbiol. Biotechnol. 64: 625-635, 2004).
Amino acid sequences used to calculate pI value of N-terminal are shown in Bold characters.

TABLE 3
Amino acid sequences, pI value of N-terminal and predicted pI
curve of representative Tat signal sequences

| | | | Length of | |
|---|---|---|---|---|
| | | | N-terminal | |
| | | | (≦10 a.a.) | |
| SEQ | | | and pI | Predicted |
| ID | Signal | | values | pI |
| Nos | sequences | Amino acid sequence | thereof | curve |
| 79 | FdnG | MDVSRRQFFKICAGGMAGTTVAALGFAPKQALA | 1-4: 3.5 | Acidic or |
| | | | 1-6: 10.75 | basic |
| 80 | FdoG | MQVSRRQFFKICAGGMAGTTAAALGFAPSVALA | 1-4: 5.75 | Neutral or |
| | | | 1-6: 12.50 | basic |
| 81 | NapG | MSRSAKPQNGRRRFLRDVVRTAGGLAAVGVALGLQQ | 1-3: 10.90 | Basic |
| | | QTARA | 1-6: 11.52 | |
| 82 | HyaA | MNNEETFYQAMRRQGVTRRSFLKYCSLAATSLGLGA | 1-3: 5.70 | Neutral or |
| | | GMAPKIAWA | 1-5: 3.09 | acidic |
| 83 | YnfE | MSKNERMVGISRRTLVKSTAIGSLALAAGGFSLPFTLR | 1-3: 9.90 | Basic |
| | | NAAA | 1-6: 9.90 | |
| 84 | WcaM | MPFKKLSRRTFLTASSALAFLHTPFARA | 1-3: 5.75 | Neutral or |
| | | | 1-5: 10.55 | basic |
| | | | 1-9: 12.52 | |
| 85 | TorA | MNNNDLFQASRRRFLAQLGGLTVAGMLGPSLLTPRR | 1-4: 5.70 | Neutral or |
| | | ATAAQA | 1-5: 3.00 | acidic |
| 86 | NapA | MKLSRRSFMKANAVAAAAAAAGLSVPGVARA | 1-2: 9.90 | Basic |
| | | | 1-6: 12.51 | |
| 87 | YebK | MDKFDANRRKLLALGGVALGAATLPTPAFA | 1-3: 6.59 | Neutral, |
| | | | 1-5: 3.91 | acidic or |
| | | | 1-10: 10.53 | basic |
| 88 | DmsA | MKTKIPDAVLAAEVSRRGLVKTTAIGGLAMASSALTL | 1-4: 10.55 | Basic |
| | | PFSRIAIIA | 1-7: 9.71 | |
| 89 | YahJ | MKESNSRREFLSQSGKMVTAAALFGTSVPLAHA | 1-3: 6.79 | Neutral or |
| | | | 1-9: 9.89 | basic |
| 90 | YedY | MKKNQFLKESDVTAESVFFMKRRQVLKALGISATAL | 1-3: 10.55 | Basic |
| | | SLPHAAHA | 1-9: 10.26 | |
| 91 | SufI | MSLSRRQFIQASGIALCAGAVPLKASA | 1-4: 5.75 | Neutral or |
| | | | 1-6: 12.50 | basic |
| 92 | YcdB | MQYKDENGVNEPSRRRLLKVIGALALAGSCPVAHA | 1-3: 5.16 | Neutral or |
| | | | 1-6: 4.11 | acidic |
| 93 | TorZ | MIREEVMTLTRREFIKHSGIAAGALVVTSAAPLPAWA | 1-5: 4.31 | Neutral or |
| | | | | acidic |
| 94 | HybA | MNRRNFIKAASCGALLTGALPSVSHAAA | 1-4: 12.50 | Basic |
| 95 | YnfF | MMKIHTTEALMKAEISRRSLMKTSALGSLALASSAFT | 1-3: 9.90 | Basic or |
| | | LPFSQMVRAAEA | 1-8: 7.64 | neutral |
| 96 | HybO | MTGDNTLIHSHGINRRDFMKLCAALAATMGLSSKAA | 1-3: 5.85 | Neutral or |
| | | A | 1-4: 3.00 | acidic |
| 97 | AmiA | MSTFKPLKTLTSRRQVLKAGLAALTLSGMSQAIA | 1-4: 5.75 | Neutral or |
| | | | 1-5: 9.90 | basic |
| | | | 1-8: 10.55 | |
| 98 | MdoD | MDRRRFIKGSMAMAAVCGTSGIASLFSQAAFA | 1-5: 12.20 | Basic |
| 99 | FhuD | MSGLPLISRRRLLTAMALSPLLWQMNTAHA | 1-8: 5.75 | Neutral or |
| | | | 1-10: 12.50 | basic |
| 100 | YedO | MTINFRRNALQLSVAALFSSAFMANA | 1-5: 5.75 | Neutral or |
| | | | 1-7: 12.50 | basic |

The above amino acids sequences of Tat signal sequences known in E. coli includes cleavage site were adopted as referenced (Tullman-Ercek et al. J. Biol. Chem., 282: 8309-8316, 2007).
Amino acid sequences used to calculate pI value of N-terminal are shown in Bold characters and twin Args are underlined.

As a result, it is confirmed that well known Sec signal sequence such as PhoA,

OmpA, StII, PhoE, MalE, OmpC, Lpp, LTB, OmpF, LamB and OmpT has basic pI value between 9.90 and 11.52 and they have common feature with the soluble expression curve at basic pI range of FIG. 1B.

In addition, since Pf3 is known as showing a strict hyperbolic shape within neutral pI range when binding to YidC (Gerken et al., Biochemistry, 47: 6052-6058, 2008) and it means that there is neutral pI range specific binding pathway, it is confirmed that this factor shares common feature with the soluble expression curve at neutral pI range of FIG. 1B. The present inventors designated this new secretion pathway as Yid pathway, since the YidC is coisolated with SecDFyajC (Nouwen and Driessen, Mol. Microbiol., 44: 1397-1405, 2002). After analyzing the N-terminal of the Pf3 which is predicted to be related to Yid pathway, we confirmed that its N-terminal has neutral pI value of 5.70 at the 1^{st} to the 6^{th} amino acids (MQSVIT, SEQ ID No: 147) and has acidic pI value of 3.30 at the 1^{st} to the 7^{th} amino acid (MQSVITD, SEQ ID No: 148). However, it is predicted that since the Yid pathway follows threading mechanism (DeLisa et al., J. Biol. Chem. 277: 29825-29831, 2002) which secrets proteins as unfolded like Sec pathway, pI value of leader peptide is important (Pf3 consists of 44 amino acids whose pI value is 6.74). In addition, after analyzing N-terminal of M13 coat protein which consists of 73 amino acids, although MKK (pI 10.55, SEQ ID No: 149) and MKKSLVLK (pI 10.82, SEQ ID No: 150) have basic pI value and thus it is the rule that the protein pass through Sec translocon like other Sec signal sequences. However, it was reported that there is no effect for the secretion in a secY mutant (Wolfe et al., J. Biol. Chem. 260: 1836-1841, 1985). With this result, we can assume that there are problems in Sec translocon by secY mutation, proteins can be secreted through Yid pathway which has near pI range. Therefore, the above Yid pathway is restricted to the secretion of relative small protein and may be an alternative pathway to Sec pathway according to intracellular situation.

Further, after analyzing pI values of N-terminals of signal sequences related to Tat pathway based on our previous reports (Korean Patent No: 981356 and Lee et al., Mol. Cells 26: 34-40, 2008) which disclose that N-terminal fragment of a signal sequence with specific pI value can substitute for whole length of the signal sequence, the present inventors confirmed that combinational length of N-terminal peptide within 10 amino acids have various range of pI, acidic to basic (Table 3). Although when the Nterminal has only one pI range, we can define the N-terminal definitely as one among acidic, neutral and basic, it is difficult to define pI range of the N-terminal when pI value of the N-terminal includes two or more ranges illustrated in FIG. 1B according to its length. However, we can acknowledge that Tat signal sequences use leader peptides with various pI values in order to secret folded proteins into the periplasm.

Even though Tat signal sequences have various acidic, neutral or basic pI ranges with a single range or with complicated ranges, considering that N-terminal with neutral pI and one with basic pI are secreted through Yid and Sec pathway, respectively, it is assumed that Tat signal sequences are secreted through Tat translocon with acidic pI value originally.

From the above result, the present inventors hypothesized that folded proteins whose signal sequences have acidic pI value are secreted through Tat pathway, ones whose signal peptides have neutral pI value are secreted through Yid pathway and ones whose signal peptides have basic pI value are secreted through Sec pathway, but exceptionally through Tat pathway. Because the diameter of Tat translocon is 70 Å (Sargent et al., Arch. Microbiol. 178: 77-84, 2002), whereas translocon related to Yid pathway participates in secreting very small proteins as describe above and thus supposed to have the smallest diameter, and SecYEG translocon has 12 Å of diameter and participates in unfolded polypeptides as chains (van den Berg et al., Nature, 427: 36-44, 2004), we can assume that the above exceptional case resulted from increment of volume of heterologous proteins fused to Sec signal peptide with basic pI value due to folding thereof. This have something to do with recent studies reporting that soluble expression of ribose binding protein having Sec signal peptide (pI of N-terminal (the 1^{st} to the 5^{th} amino acids) is 10.55) is enhanced with tatABC operon (Pradel et al., BBRC, 306: 786-791, 2003) and reporting that soluble expression of L2 -lactamase (pI of N-terminal (the 1^{st} to the 6^{th} amino acids) is 12.80) is related to tatC (Pradel et al., Antimicrob. Agents Chemother., 53: 242-248, 2009).

Therefore the present inventors acknowledged that unfolded proteins are secreted through Tat pathway when signal sequences have N-terminals with acidic pI value, through Yid pathway when the signal sequences have N-terminals with neutral pI value, and through Sec pathway when the signal sequences have N-terminals with basic pI value. In addition, the present inventors acknowledged that folded bulky proteins are secreted through Tat pathway because they get larger volume regardless of pI value of N-terminal of their signal sequence. Thus, present inventors suggest a schematic diagram regarding secretional pathways classifying the E. coli type-II periplasmic secretion pathway into three categories, Sec, Yid and Tat (FIG. 2).

### Example 3

### Analysis of Effect of pI Value and Hydrophilicity of Leader Peptides on Soluble Expression of GFP

The present inventors predicted that GFP, a bulky folded active protein will be secreted through Tat pathway and it will possible to enhance the secretion of GFP by a leader peptide whose pI value is acidic and whose hydrophilicity is high to that of N-terminal of the GFP, based on the result of Example 2 in that a protein whose N-terminal has acidic pI value is secreted through Tat pathway and even though a signal peptide is one using the other secretional pathway such as Sec pathway and Yid pathway, when a secreted protein is a bulky folded active protein the protein is secreted through Tat pathway.

<3-1> Construction of GFP Expression Vectors and Analyses of Soluble Expression

In order to construct GFP expression vectors, a PCR reaction was performed with forward primers having nucleotide sequences of SEQ ID Nos: 123 to 141 and 143 to 145 comprising NdeI recognition site (CAT ATG) at 5-end and a reverse primer having nucleotide sequence of SEQ ID No: 146 which deletes the stop codon TAA and comprising XhoI recognition site (CTC GAG) using GFP ORF as a template and then the PCR product was cloned to NdeI-Xhol site of pET-22b(+) resulting in the construction of pET-22b(+) (N-terminal-gfp-XhoI-His tag) expression vector. pET-22b(+) (gfp-XhoI-His tag) expression vector was used as a control. In addition, in order to construct TorAss-GFP clone having TorA signal sequence (Mejean et al., Mol. Microbiol. 11: 1169-1179, 1994), one of Tat signal sequences as a control, a first PCR reaction was performed with a forward primer having nucleotide sequences of SEQ ID No: 142 (TorAss₂₀₋₃₉-agaa-GFP₁₋₇) and a reverse primer having nucleotide sequence of SEQ ID No: 146 using pEGFP-N2 vector, a GFP expression vector as a template. And then the first PCR product was used as a template for a second PCR reaction. The second PCR reaction was performed with a forward primer having nucleotide sequences of SEQ ID No: 143 (TorAss₁₋₂₇) and a reverse primer having nucleotide sequence of SEQ ID No: 146 and the second PCR product was cloned into pET-22b(+) vector. The GFP protein used in the present example was confirmed as one having several transmembrane-like domains by analyzing hydrophilicity according to Hopp-Woods scale.

E. coli BL21(DE3) was transformed with the expression vectors constructed above using a conventional method and the transformants were cultured in LB media (Tryptone 20 g/L, yeast extract 5 g/L, NaCl0.5 g/L, KCl 1.86 mg/L) with 100 μg/L ampicillin overnight at 30 C and then the culture was diluted 100 times with LB media and cultured until OD₆₀₀ is 0.3. And then, 1 mM IPTG was added for induction and was further cultured for 3 hr. One ml of the culture was centrifuged at 4,000 g for 30 min at 4C and wet weight of pellet was measured for fluorescent assay before resuspending the pellet with 100 to 200 μl of 50 mM Tris buffer (pH 8.0).The suspension was sonicated with 152-s cycle pulses (at 30% power output) in order to isolate total protein fraction and then the sonicated solution was centrifuged at 16,000 rpm for 30 min at 4 C and supernatant was isolated as soluble fraction. Fluorescence of a fixed quantity of total protein fraction and corresponding soluble fraction was detected using a fluorescent analyzer (Perkin Elmer Victor3, USA) at an excitation wavelength of 485 nm and an emission wavelength of 535 nm, respectively (FIG. 3C). 50 μg of proteins per well were loaded on 15% SDS-PAGE gel and SDS-PAGE analyses were performed according to Laemmli (Nature, 227: 680-685, 1970). The gels were stained with Coomassie Brilliant Blue stain (Sigma, USA). In the meantime the gels after SDS-PAGE analyses were transferred to Hybond-P membrane; GE, The extent of expression of the recombinant GFP was quantified using anti-His tag antibody as a primary antibody and alkaline phosphatase-conjugated anti-mouse antibody was used as a secondary antibody. Finally the recombinant GFP was detected with a chromogenic Western blotting kit (Invitrogen, USA) according to manufacturer's instruction (FIG. 3A and 3B).

<3-2> Analysis of Effect of pI Value of N-Terminal of a Signal Peptidevariant on Soluble Expression of GFP

In order to analyze effect of pI value of N-terminal of signal peptide on soluble expression of GFP, the present inventors investigated the extent of soluble expression of GFP linked to leader peptides consisting of variant of OmpA signal peptide whose N-terminal pI value is adjusted and hydrophilic Arg polymer rather than using twin Arg motif which is a conservative region in Tat pathway signal sequence. For this purpose, the present inventors used GFP expressed from pET-22b(+)(gfp-XhoI-His tag) constructed by cloning of gfp region of pEGFP-N2 vector into NdeI-XhoI site of pET-22b(+) as described in Example 3-1. That is, the leader peptides consisting of variants of OmpASP₁₋₈ (M(X)(Y) in which pI value of N-terminal of OmpASP₁₋₈ is empirically adjusted except the first amino acid Met) and a hydrophilic Arg polymer were designed as M(X)(Y)-TAIAI(OmpASP₄₋₈)-8Arg and then pI value of M(X)(Y) and the hydrophilicity of M(X)(Y)-TAIAI(OmpASP₄₋₈)-8Arg were measured (Table 4).

The present inventors investigated GFP expression level by transforming E. coli BL21(DE3) with the constructed GFP expression vector using the method described in Example 3-1. As a result, when the leader peptide has N-terminal of MEE (pI 3.09, SEQ ID No: 7) which belongs to acidic pI range, higher expression level than control was observed; when the leader peptide has N-terminal of MAA (pI 5.60, SEQ ID No: 13) and MAH (pI 7.65, SEQ ID No: 18), which belong to neutral pI range, higher or lower expression level than control was observed; and when the leader peptide has N-terminal of MKK (pI 10.55, SEQ ID No: 149) and MRR (pI 12.50, SEQ ID No: 151) which belong to basic pI range, little expression level was observed (FIG. 3). However even though the N-terminal of the leader peptide is MKK or MRR somewhat fluorescent was detected in total protein fraction thus it was confirmed that some amount of GFP exists in cytosol whereas little fluorescent was detected in soluble fraction. Thus it is assumed that GFP whose N-terminal is MKK or MRR has difficulty to pass through Sec translocon which is relative narrow. This result is interpreted that GFP binds to proteins associated to transmembrane proteins thus was not detected in Western blot analysis, as shown that GFP bands of total protein fraction and soluble fraction were seen as smear appearance upper position than that of control (FIG. 3).

Therefore, the present inventors acknowledged that bulky folded heterologous proteins may be secreted through Tat pathway when a leader peptide consisting of an OmpA signal peptide fragment variant whose N-terminal pI value is adjusted to acidic and neutral range and hydrophilic Arg polymer is fused thereto.

In addition, the present inventors confirmed that pI value of N-terminal of a leader peptide has strong effect on the selection of transmembrane channel and Sec pathway which is different from Tat pathway from the result that when a leader peptide consisting of an OmpA signal peptide fragment variant whose N-terminal pI value is adjusted to basic range and hydrophilic Arg polymer is fused thereto, it is difficult to secrete GFP because the GFP, a bulky folded protein has channel selectivity on Sec transmembrane channel and thus it should path through the Sec channel relative narrow to Tat channel.

Further, it is assumed that a leader peptide with neutral pI value can induce the secretion of a heterologous protein linked thereto through Tat pathway without attenuation as seen in Sec pathway, since the leader peptide may have weak channel selectivity on Yid pathway corresponding thereto or the heterologous protein may not pass through the Yid pathway because Yid translocon may have narrower diameter than Sec translocon, from the result that GFP having a leader peptide with neutral pI value was somewhat well secreted although the extent of soluble expression was lower than that of GFP having a leader peptide with acidic pI value and no inhibition of soluble expression through Yid pathway was not observed. It is assumed that when a protein having larger molecular weight is folded, it will be secreted through Tat translocon without blocking through Yid pathway due to the large volume of the folded protein than the diameter of the Yid translocon since the blocking phenomenon shown in Sec pathway may be due to GFP consisting of relative small number of amino acids (239 amino acids), whose size is slightly bigger to cause blocking, but not much bigger to prevent blocking than the diameter of the Sec translocon. In addition, the above result is coincident with the result that leader peptides and secretional enhances of MEE (pI 3.09, SEQ ID No: 7), MAA (pI 5.60, SEQ ID No: 13), MAH(pI 7.65)-OmpASP₄₋₁₀-6Arg (SEQ ID No: 152) or MEE(pI 3.09)-OmpASP₄₋₁₀-6Glu (SEQ ID No: 153) induced soluble expression of Olive flounder hepcidin I (Korean Patent Gazette No: 2009-0055457).

From the above result that when a leader peptide of GFP, a bulky folded active protein, has N-terminal with acidic or neutral pI value, the GFP was secreted through Tat pathway, when the leader peptide has N-terminal with basic pI value, the GFP blocked Sec translocon passing therethrough, the present inventors confirmed that the suggestion that soluble secretional pathway is determined according to pI value of N-terminal of a protein and all the bulky folded proteins are secreted through Tat pathway is reasonable (FIG. 2).

<3-3> Analysis of Effect of Met-Hydrophilic Amino Acid Sequence and G_{RNA} Value on Soluble Expression of GFP

<3-3-1> Analysis of Effect of Met-Hydrophilic Amino Acid Sequence on Soluble Expression of GFP

In order to investigate effect of hydrophilic amino acids linked to methionine (Met) as a leader peptide on soluble expression of GFP, the present inventors designed leader peptides which sequentially consisting of Met and 6 homotype hydrophilic amino acids linked thereto and constructed expression vectors expressing the leader peptides and GFP fused thereto. E. coli BL21(DE3) was transformed with the expression vectors using the method described in Example 3-1 and expression level of GFP was determined (FIG. 4.). The homotype hydrophilic amino acids were selected from a group consisting of Asp, Glu, Lys and Arg, and pI value and hydrophilicity corresponding thereto were analyzed (Table 4).

As a result, GFPs having MDDDDDD (pI 2.56, hy 1.82, SEQ ID No: 106) and

MEEEEEE(pI 2.82, hy 1.82, SEQ ID No: 107) with acidic pI value and high hydrophilicity as leader peptides showed high level of soluble expression, MEEEEEE among them showed the highest soluble expression level. From these results, it is assumed that soluble expression of bulky folded GFP may be mediated by Tat pathway when MDDDDDD or MEEEEEE which are hydrophilic leader peptide having N-terminal with acidic pI are linked to the GFP.

However in the case of leader peptides having N-terminal with basic pI value, a leader peptide MRRRRRR (pI 13.20, hy 1.82, SEQ ID No: 109) did not induce soluble expression of GFP whereas a leader peptide MKKKKKK (pI 11.21, hy 1.82, SEQ ID No: 108) showed high level of expression of active GFP.

The case of MKKKKKK, high level of expression and fluorescence in total protein fraction continued to those in soluble fraction, and thus it seems that the folded bulky GFP was secreted through Tat translocon rather than Sec pathway. Therefore, it is coincident with the suggestion of the present inventors that a leader peptide having N-terminal with basic pI value should pass through Tat pathway if a folded protein has larger volume (FIG. 2).

Although the result that MRRRRRR which is predicted to have similar result to

MKKKKKK indeed inhibited soluble expression of GFP is not coincident with our prediction, all clones constructed to express GFP fusion protein having leader peptides MRRRRRR (pI 13.20, hy +1.82), MRRRRRRRRR (pI 13.40, hy +2.17, SEQ ID No: 110) and MRRRRRRRRRRRR (pI 13.54, hy +2.36, SEQ ID No: 111) have very little expression level of GFP after Western blot analysis on whole protein fraction. Thus, from the result of MKKKKKK whose high level of soluble expression and fluorescence in whole protein fraction continued to those in soluble fraction, the extent of soluble expression of a heterologous protein having N-terminal with basic pI and high hydrophilicity is dependent on expression level of the heterologous protein among whole proteins.

Consequently, it was confirmed that a bulky folded heterologous protein linked to a leader peptide having an N-terminal with acidic or basic pI value and comprising high hydrophilicity was secreted through Tat pathway in a folded form. Particularly, when the leader peptide has both basic pI value in its N-terminal and highly hydrophilic amino acids, the selectivity on Sec channel is weaken, and there is critical difference in the selection of secretional channel from a leader peptide having an anchor function space, TAIAI (OmpASP₄₋₈) consisting of amino acids not effecting pI value of the leader peptide between the N-terminal and the hydrophilic amino acids as shown in Example 3-2.

In addition, from the result, the secretion of bulky folded GFP linked to a leader peptide consisting of a basic N-terminal, an anchor function space and hydrophilic amino acids such as MKK(OmpASP₁₋₃, pI 10.55)-TAIAI(OmpASP₄₋₈)-8Arg (SEQ ID No: 104) and MRR(pI 12.50)-TAIAI(OmpASP₄₋₈)-8Arg (SEQ ID No: 105) through Sec translocon was inhibited because the N-terminal of the leader peptide maintained a function as an anchor to the Sec translocon (FIG. 3), it was confirmed that the leader peptides are Sec translocon-specific leader peptides and the difference in channel selection was due to characteristic of the leader peptide, folding state, size of a heterologous protein linked thereto.

<3-3-2> Analysis Effect of Total Expression Level in Leader Peptides Having N-Terminals with Basic pI Value and High Hydrophilicity on Soluble Expression of GFP

From the result of Example 3-3-1, the present inventors confirmed that there are other key factors for soluble expression besides pI value and hydrophilicity. Thus the present inventors analyzed G_{RNA} value of polynucleotides consisting of translation initiation region of pET-22b(+) vector and MKKKKKK-GFP₁₋₅ or MRRRRRR-GFP₁₋₅ encoding regions (SEQ ID No: 155, 5′-AAG AAG GAG ATA TAC AT-ATG AAA AAA AAA AAA AAA AAA-ATG GTG AGC AAG GGC-3′; or SEQ ID No: 156, 5′-AAG AAG GAG ATA TAC AT-ATG CGT CGC CGT CGC CGT CGC-ATG GTG AGC AAG GGC-3′, respectively), in order to investigate whether the difference of soluble expression between MKKKKKK and MRRRRRR which are leader peptides having similar pI value and hydrophilicity is due to translation efficiency. MFOLD 3 software (Zuker, Nucleic Acids Res. 31: 3406-3415, 2003) was used for calculating G_{RNA} value. If there are several G_{RNA} values for a RNA molecule, it means that there may be several secondary structures. However, the lower G_{RNA} values the RNA molecule has the more stable secondary structure it has.

As a result, the present inventors confirmed that G_{RNA} values at the position described above of MKKKKKK is 0.60 and 1.60 and that of MRRRRRR is −13.80, thus two clones are very different from each other and it is acknowledged that an RNA encoding MRRRRRR has more stable secondary structure than one encoding MKKKKKK because the former has less G_{RNA} value than the latter.

In addition, the present inventors constructed GFP fusion clones using polypeptides encoding leader peptides of MKKRKKR-I(Lys^{AAA}Lys^{AAA}Arg^{CGC})₂ (G_{RNA} −1.00, −0.50, −0.30, SEQ ID No: 112), MKKRKKR-II(Lys^{AAG}Lys^{AAA}Arg^{CGC})₂(G_{RNA} −1.00, −0.50, −0.30, SEQ ID No: 113)_{and MRRKRRK}(Arg^{CGT}Arg^{CGC}Lys^{AAA})₂(G_{RNA} −7.60, SEQ ID No: 114), which are variants of MKKKKKK(Lys^{AAA})₆ (G_{RNA} 0.60, 1.60, SEQ ID No: 108) and MRRRRRR(Arg^{CGT}Arg^{CGC})₃(GRNA −13.80, SEQ ID No: 109), having same hydrophilicity therewith (Table 4) and then analyzed the extent of soluble expression of the GFP fusion clones (FIG. 5). The MKKKKKK(Lys^{AAA})₆ and MRRRRRR(Arg^{CGT}Arg^{CGC})3 clones were used as controls.

As a result, there is no difference between MKKKKKK and MKKRKKR-I in soluble expression. However MKKRKKR-I and -II having same G_{RNA} value showed noticeable difference in the extent of soluble expression, and MRRKRRK(Arg^{CGT}Arg^{CGC}Lys^{AAA})₂ which has relative low G_{RNA} value showed somewhat high level fluorescence. Clones showing the correlation between the expression level of GFP and G_{RNA} value, and clones not showing the correlation coexist and MKKRKKR-I and -II showed remarkable difference even though they have same G_{RNA} value. However it seems that this remarkable difference is due to codon wobble phenomenon (Lee et al., Mol. Cells, 30:127-135, 2010) against anticodon UUU for Lys between Lys^{AAA} and Lys^{AAG}. Thus, excluding exceptional cases due to wobble phenomenon, the G_{RNA} value may be a criterion for expression level of a heterologous protein.

In addition, since GFP expression level in total protein fraction was correlated to the extent of soluble expression of GFP and hydrophilicity was related to the secretion of GFP consistently, it is acknowledged that total translational level of a heterologous protein having N-terminal with basic pI value and comprising a plurality of hydrophilic amino acids is correlated to soluble expression of the heterologous protein.

Further, the above phenomenon may be applied to a leader peptide having N-terminal with acidic and basic pI value and comprising a plurality of hydrophilic amino acids, and total translational level of a heterologous protein fused to the leader peptide may be connected to soluble expression. That is, the secretion of a heterologous protein through Tat pathway may be dependent on channel selectivity and total translational efficiency of the heterologous protein. Thus, it is important to design a leader peptide having N-terminal with acidic or neutral pI in order to enhance soluble expression of the heterologous protein when the heterologous protein is a bulky folded active protein. In addition, if one chooses a leader peptide having N-terminal with basic pI, it is important to design a polynucleotide encoding the leader peptide and N-terminal of a heterologous protein with high G_{RNA} value as well as to design the leader sequence in order to obviate Sec pathway, which tends to be blocked with basic N-terminal of the leader peptide.

Although the leader peptide MRRRRRR (SEQ ID No: 109) did not induce moderately soluble expression of GFP, an interaction between a leader peptide and a characteristic of a heterologous protein linked thereto seems to be correlated to soluble expression of the heterologous protein, from the result of Korean Patent Gazette No: 2009-0055457 which discloses that leader peptides MKKKKKKK (SEQ ID No: 157) and MRRRRRRR (SEQ ID No: 158) induced soluble expression of Olive flounder hepcidin I successfully.

<3-4> Analysis of Effect of Modification of N-Terminal of GFP on Soluble Expression of GFP

From the previous result, the inventors recognized that a leader peptide MEEEEEE

(SEQ ID No: 107) induced the highest level of soluble expression of GFP (FIG. 4, lane 3). The present inventors constructed GFP expression vectors comprising polynucleotides encoding modified GFP whose one or more amino acids among the 2^{nd} to the 5^{th} position was substituted with a hydrophilic amino acid, Glu, transformed E. coli BL21(DE3) with the expression vectors using a method described Example 3-1, and determined GFP expression level in total protein fraction and soluble fraction in order to investigate whether the modification of N-terminal of a heterologous protein effects on soluble expression of GFP (FIG. 6). The above GFP expression vectors were designated as GFP₁₋₇(V2E) (SEQ ID No: 116), GFP₁₋₇(V2E-S3E) (SEQ ID No: 117), GFP₁₋₇(V2E-S3E-K4E) (SEQ ID No: 118) and GFP₁₋₇(V2E-S3E-K4E-G5E) (SEQ ID No: 119), respectively, and pI values and hydrophilicities thereof were analyzed (Table 4 and FIG. 6).

Consequently, clones having GFP₁₋₇(V2E), GFP₁₋₇(V2E-S3E) or GFP₁₋₇(V2E-S3E-K4E) showed higher level of soluble expression than control. Particularly, V2E made by substitution of the 2nd valine followed by the 1^{st} Met with glutamate, which showed the highest level of soluble expression and GFP₁₋₇(V2E-S3E-K4E-G5E) whose hydrophilicity is highest showed little lower level of soluble expression than control (FIG. 6, lane 5). From the above result, it is acknowledged that pI value according to the position where a hydrophilic amino acid is inserted at the N-terminal correlates to soluble expression of GFP rather than just only hydrophilicity if the hydrophilicity is over certain degree, although the more hydrophilic amino acids such as glutamate are added, the higher the level of soluble expression of GFP gets generally.

TABLE: 4
Soluble expression level of GFP according to amino acid sequences,
pI values and hydrophilicities

| | Amino acid | | | | | |
|---|---|---|---|---|---|---|
| | sequences of | | | | | Relative |
| SEQ | N-terminal | | | SEQ | | soluble |
| ID | of leader | pI | | ID | Forward primers used for designing leader | expres- |
| Nos | peptides | value | Hy* | Nos | peptides | sion |
| 101 | MEE-TAIAI- | 3.09 | 1.34 | 123 | CAT ATG GAA GAG ACA GCT ATC GCG ATT [symbol] [symbol] [symbol] | ++ |
| | 8 × Arg | | | | [symbol] [symbol] [symbol] [symbol] [symbol] ATG GTG AGC AAG GGC GAG GAG | |
| 102 | MAA-TAIAI- | 5.60 | 1.16 | 124 | CAT ATG GCT GCA ACA GCT ATC GCG ATT [symbol] [symbol] [symbol] | + |
| | 8 × Arg | | | | [symbol] [symbol] [symbol] [symbol] [symbol] ATG GTG AGC AAG GGC GAG GAG | |
| 103 | MAH-TAIAI- | 7.65 | 1.16 | 125 | CAT ATG GCT CAC ACA GCT ATC GCG ATT [symbol] [symbol] [symbol] | + |
| | 8 × Arg | | | | [symbol] [symbol] [symbol] [symbol] [symbol] ATG GTG AGC AAG GGC GAG GAG | |
| 104 | MKK-TATAI- | 10.55 | 1.34 | 126 | CAT ATG AAA AAA ACA GCT ATC GCG ATT [symbol] [symbol] [symbol] | − |
| | 8 × Arg | | | | [symbol] [symbol] [symbol] [symbol] [symbol] ATG GTG AGC AAG GGC GAG GAG | |
| 105 | MRR-TAIAI- | 12.50 | 1.34 | 127 | CAT ATG CGT CGC ACA GCT ATC GCG ATT [symbol] [symbol] [symbol] | − |
| | 8 × Arg | | | | [symbol] [symbol] [symbol] [symbol] [symbol] ATG GTG AGC AAG GGC GAG GAG | |
| 106 | M-D6 | 2.56 | 1.82 | 128 | CAT ATG [symbol] [symbol] [symbol] [symbol] [symbol] [symbol] ATG GTG AGC AAG | ++ |
| | | | | | GGC GAG GAG | |
| 107 | M-E6 | 2.82 | 1.82 | 129 | CAT ATG [symbol] [symbol] [symbol] [symbol] [symbol] [symbol] ATG GTG AGC AAG | ++++++ |
| | | | | | GGC GAG GAG | |
| 108 | M-K6 | 11.21 | 1.82 | 130 | CAT ATG [symbol] [symbol] [symbol] [symbol] [symbol] [symbol] ATG GTG AGC AAG | ++++ |
| | | | | | GGC GAG GAG | |
| 109 | M-R6 | 13.20 | 1.82 | 131 | CAT ATG [symbol] [symbol] [symbol] [symbol] [symbol] [symbol] ATG GTG AGC AAG | − |
| | | | | | GGC GAG GAG | |
| 110 | M-R9 | 13.40 | 2.17 | 132 | CAT ATG [symbol] [symbol] [symbol] [symbol] [symbol] [symbol] [symbol] [symbol] [symbol] ATG | − |
| | | | | | GTG AGC AAG GGC GAG GAG | |
| 111 | M-R12 | 13.54 | 2.36 | 133 | CAT ATG [symbol] [symbol] [symbol] [symbol] [symbol] [symbol] [symbol] [symbol] [symbol] [symbol] | − |
| | | | | | [symbol] [symbol] ATG GTG AGC AAG GGC GAG GAG | |
| 112 | MKKRKKR-I | 12.53 | 1.82 | 134 | CAT ATG [symbol] [symbol] [symbol] [symbol] [symbol] [symbol] ATG GTG AGC AAG | ++++ |
| | | | | | GGC GAG GAG | |
| 113 | MKKRKKR-II | 12.53 | 1.82 | 135 | CAT ATG [symbol] [symbol] [symbol] [symbol] [symbol] [symbol] ATG GTG AGC AAG | + |
| | | | | | GGC GAG GAG | |
| 114 | MRRKRRK | 12.98 | 1.82 | 136 | CAT ATG [symbol] [symbol] [symbol] [symbol] [symbol] [symbol] ATG GTG AGC AAG | +++ |
| | | | | | GGC GAG GAG | |
| 115 | GFP₁₋₇ | 4.31 | 1.06 | 137 | CAT ATG GTG AGC AAG GGC GAG GAG | + |
| | (control) | | | | | |
| 116 | GFP₁₋₇ | 4.01 | 1.27 | 138 | CAT ATG [symbol] AGC AAG GGC GAG GAG CTG TTC ACC GGG | ++++ |
| | (V2E) | | | | GTG | |
| 117 | GFP₁₋₇ | 3.84 | 1.46 | 139 | CAT ATG [symbol] [symbol] AAG GGC GAG GAG CTG TTC ACC GGG | +++ |
| | (V2E-S3E) | | | | GTG | |
| 118 | GFP₁₋₇ | 2.87 | 1.46 | 140 | CAT ATG [symbol] [symbol] [symbol] GGC GAG GAG CTG TTC ACC GGG | ++ |
| | (V2E- | | | | GTG | |
| | S3E-K4E) | | | | | |
| 119 | GFP₁₋₇ | 2.82 | 1.82 | 141 | CAT ATG [symbol] [symbol] [symbol] [symbol] GAG GAG CTG TTC ACC GGG | + |
| | (V2E- | | | | GTG | |
| | S3E-K4E- | | | | | |
| | G5E) | | | | | |
| 120 | TorAss- | N.T | N.T | 142 | TTA ACC GTC GCC GGG ATG CTG GGG CCG TCA TTG TTA | N.T |
| | GFP₁₋₇ | | | | ACG CCG CGA CGT GCG ACT GCG GCG CAA GCG GCG ATG | |
| | (control) | | | | GTG AGC AAG GGC GAG GAG | |
| | | | | | (TorAss₂₀₋₃₉-aqaa-GFP₁₋₇) (primary primer) | |
| | | | | 143 | CAT ATG AAC AAT AAC GAT CTC TTT CAG GCA TCA CGT | + |
| | | | | | CGG CGT TTT CGT GCA CAA CTC GGC GGC TTA ACC GTC | |
| | | | | | GCC GGG ATG CTG (Tor Ass₁₋₂₇) (secondary primer) | |
| 121 | OmpASP₁₋₃- | 10.55 | N.T | 144 | CAT ATG [symbol] [symbol] ACA GCT ATC GCG ATT GCA GTG GCA | +/− |
| | OmpAss₄₋₂₃ | | | | CTG GCT GGT TTC GCT ACC GTA GCG CAG GCC GCT CCG | |
| | (control) | | | | ATG GTG AGC AAG GGC GAG GAG | |
| 122 | MKKKKKK(pI | 11.21 | 1.82 | 145 | CAT ATG [symbol] [symbol] [symbol] [symbol] [symbol] [symbol] ACA GCT ATC GCG | +/− |
| | 11.21, hy | | | | ATT GCA GTG GCA CTG GCT GGT TTC GCT ACC GTA GCG | |
| | 1.82)- | | | | CAG GCC GCT CCG ATG GTG AGC AAG GGC GAG GAG | |
| | OmpAss₄₋₂₃ | | | | | |

| | | | |
|---|---|---|---|
| Reverse primer | 146 | CTC GAG CTT GTA CAG CTC GTC CAT GCC | N.T |

Hy is an abbreviation for hydrophilicity and was calculated by DNASIS ™ software according to Hoop-Woods scale (window size: 6 and threshold line: 0.00). If the hydrophobicity value is +, the peptide is hydrophilic, while if the hydrophobicity is −, the peptide is hydrophobic.
Bold characters in amino acid sequences refer to regions used for the calculation of pI value.
TAIAI refers to OmpASP₄₋₈ (Korean Patent No: 981356).
OmpAss refers to a full-length OmpA signal sequence (OmpASP₁₋₂₁ + OmpA₁₋₂, Korean Patent No: 981356).
Hydrophilicities were calculated with amino acid sequence of N-terminal of leader peptide listed in the second column.
CAT refers to an extended nucleotides for conserving Nde I site.
Bold characters in nucleotide sequences refer to polynucleotides effecting pI values of signal peptide variants.
Bold italic characters refer to polynucleotides corresponding to amino acids related to various pI values and hydrophilicities.
Bold underlined characters refer to polynucleotides corresponding to substituted amino acids.
Normal characters refer to polynucleotides corresponding GFP encoding region (pEGFP-N2 vector, Clontech).
Italic characters refer to polynucleotides corresponding OmpA and T or A signal sequence.
Reverse primer refers to a complementary nucleotide sequence to a polynucleotide comprising region corresponding to C-terminal of GFP, Xho I site and a region corresponding His tag of pET-22b(+).
N.T refers to “not tested”.

In this case, pI value of GFP₁₋₇(V2E) was 3.25 when calculated for ME and 4.01 when calculated for MESKGEE (SEQ ID No: 116) whereas pI value for GFP₁₋₇(V2E-S3E-K4E-G5E) (MEEEEEE, SEQ ID No: 119) was calculated as 2.82 which is pI value of whole sequence MEEEEEE because all glutamate are connected to one another thus it is difficult to isolate amino acids effecting pI value. Regarding these soluble expression levels according to pI value of N-terminal, it is confirmed that expression patterns at N-terminal pI value of 3.25 and 4.01 is correlated to relative high soluble expression pattern of rMefp1 having leader peptides with N-terminal pI value of 3.25 to 4.61 shown in FIG. 1B, Table 1 and FIG. 2, and expression patterns at N-terminal pI value of 2.82 is correlated to relative low soluble expression pattern of rMefp1 having a leader peptide with N-terminal pI value of 2.82 shown in FIG. 1B, Table land FIG. 2.

In addition, although GFP₁₋₇(V2E-S3E) and GFP₁₋₇(V2E-S3E-K4E) has same hydrophilicities before GFP₅₋₇, they have different pI values (MEEK, pI 4.31 and MEEE, pI 2.99) and showed remarkable difference in the extent of soluble expression of GFP. Thus, regarding the difference in the extent of soluble expression of GFP, it is recognized that the expression pattern at N-terminal pI value of 4.31 is correlated to relative high soluble expression pattern of rMefp1 having leader peptides with N-terminal pI value of 3.25 to 4.61 shown in FIG. 1B, Table 1 and FIG. 2, and expression patterns at N-terminal pI value of 2.99 is correlated to relative low soluble expression pattern of rMefp1 having a leader peptide with N-terminal pI value of 2.92 to 3.09 shown in FIG. 1B, Table land FIG. 2

Further, although MEEEEEE (SEQ ID No: 107) and GFP₁₋₇(V2E-S3E-K4E-G5E) (SEQ ID No: 119) have the same pI value and hydrophilicity, GFP₁₋₇(V2E-S3E-K4E-G5E) in which GFP₈₋₁₄(LFTGVVP, pI 5.85, by -0.58, SEQ ID No: 152) is linked to MEEEEEE showed lower soluble expression level than control whereas MEEEEEE in which GFP₁₋₇(MVSKGEE, pI 4.31, by +1.06, SEQ ID No: 115) is linked thereto showed higher soluble expression than control. From the result, although a leader peptide has the same N-terminal pI and hydrophilicity, it is acknowledged that the hydrophilicity of successive amino acids strongly affects on the soluble expression of a heterologous protein

Therefore, one can recognize that it is possible to enhance the expression and the secretion of a bulky folded heterologous protein through Tat pathway by substituting several amino acids with acidic or neutral but hydrophilic amino acids in N-terminal of the bulky folded heterologous protein thereby adjusting pI value and hydrophilicity thereof and optimizing the expression condition and that the closer the substituted amino acids are to the N-terminal, the stronger effect the substitution has. It is suggested that other homotype or heterotype amino acids may be applied to induce high level of soluble expression by adjusting pI value and hydrophilicity of a leader peptide of a bulky folded active protein from the present example.

<3-5> Analysis of Effect of High Hydrophilicity of N-Terminal in a Signal Peptide/Sequence on Soluble Expression of GFP

The present inventors constructed an expression vector, MKKKKKK-OmpAss₄₋₂₃ (SEQ ID No: 122)-GFP (N-terminal: MKKKKKK, pI 11.21) and a control, OmpAss₁₋₂₃ (SEQ ID No: 121)-GFP (N-terminal: MKK, pI 10.55) using a relatively short length fragment of OmpA signal peptide (Korean Patent No: 981356) and determined soluble expression level by the method described in Example 3-1 (Table 4 and FIG. 7), in order to investigate whether high hydrophilicity of signal peptide N-terminal affects on soluble expression of GFP from the result of Examples 3-3 and 3-4 which disclose that a leader peptide having N-terminal with acidic or basic pI value and high hydrophilicity enhanced soluble expression of GFP.

As a result, expression of GFP in total protein fractions of both the clones with

Western blot analysis were good but the fluorescent levels thereof quite lower than that of TorAss-GFP used as another control. Expressions of GFP in soluble fractions of both the clones were lower than that of control TorAss-GFP and the fluorescent levels thereof were very low too. The Fluorescent level of MKKKKKK-OmpAss₄₋₂₃-GFP was little higher than that of the control OmpAss₁₋₂₃-GFP, but it is lower than that of another control, TorAss-GFP. Thus, it is recognized that high hydrophilicity of signal peptide N-terminal is not effective for soluble expression of GFP from the result that the MKKKKKK-OmpAss₄₋₂₃-GFP showed lower soluble expression level than a clone having only MKKKKKK (SEQ ID No: 108) as a leader peptide (FIG. 7, lane 5), although hydrophilicity of signal peptide N-terminal was increased.

It is thought that the above consequences resulted from the inhibition of the secretion into the periplasm of a heterologous protein by binding of SecA protein which binds to central hydrophobic region (Wang et al., J. Biol. Chem. 275: 10154-10159, 2000) and signal peptidase which binds to C-terminal cleavage site of a signal peptide thereto, although elevating hydrophilicity of the N-terminal of the heterologous protein when a Sec signal peptide is used. Thus, it is assumed that N-terminal having basic pI value and high hydrophilicity within a Sec signal sequence will be less effective to induce soluble expression than an independent leader peptide having basic pI value and high hydrophilicity without common regions of the Sec signal sequences.

In addition, it assumed that a folding process of a bulky folded heterologous protein using Tat signal peptides in the cytosol will be inhibited by binding of proteins which bind to hydrophobic and cleavage region of the signal peptides (FIG. 7, see low molecular weight band of lane 2) because the Tat signal peptides have N-terminal region, a central hydrophobic region and a C-terminal cleavage region. Further, considering the characteristic of Tat translocon that there is no folding process in the periplasm (see below), the activity of the heterologous protein will decline although it would be secreted into the periplasm. Therefore, it is assumed that N-terminal having acidic pI value and high hydrophilicity within a Tat signal sequence will be less effective to induce soluble expression than an independent leader peptide having acidic pI value and high hydrophilicity without common regions of the Tat signal sequences.

In the case of TorA signal sequence, control TorAss-GFP showed both primitive GFP (upper band) form and mature GFP form (lower band) in soluble fraction (FIG. 7B, lane 2 and FIG. 6B, lane 6) but the soluble fraction has only ⅓ to ½ of fluorescent compared to control GFP (FIG. 6C and FIG. 7C) although the band areas of the soluble GFP are similar to that of control GFP (FIG. 6B, lane 6 and FIG. 7B, lane 2). It is acknowledged that mature GFP (lower band) in which a signal peptide is deleted by a signal peptidase does not emit sufficient fluorescence although primitive TorAss-GFP emits fluorescence from the result. It is assumed that TorAss-GFP which is a primitive form of a heterologous protein having Tat signal peptide such as TorA signal sequence passes through in folded form and emits fluorescence, but mature GFP whose TorA signal peptide is deleted by a signal peptidase is secreted but folding process is inhibited by binding of the signal peptidase in cleavage processing and the secreted protein which is partially folded or not folded any more in the periplasm thus emits weak fluorescence.

However, GFP having OmpA signal sequence (FIG. 7, lane 3), one of Sec signal sequences as a leader peptide and GFP having MKKKKKK-OmpAss₄₋₂₃ as a leader peptide (FIG. 7, lane 4) emitted weak fluorescence although they showed high level of expression in total protein fraction. Thus, it assumed that a signal peptidase inhibited folding process. In addition, since the both proteins showed relatively low expression level in soluble fraction, it seems that both the GFPs emit weak fluorescence because they are secreted into the periplasm as unfolded forms through Sec translocon with diameter of about 12 Å and folded in the periplasm regardless their forms, primitive or mature.

Therefore, it is assumed that a heterologous protein selecting through the Sec pathway cannot pass through the Sec pathway when the secretion process is relative slow and the original protein is folded thereby, while the secretion via Sec translocon is induced by the formation of a mature protein which is unfolded by binding of a signal peptidase to the immature protein and then the unfolded mature protein secreted into the periplasm and folded in the periplasm.

However, it is assumed that GFP having a Tat signal peptide emits fluorescent by passing Tat translocon in a primitive folded form and a mature GFP whose signal peptide is cleaved and secreted into the periplasm through the Tat translocon is unfolded whereby the folding process is partially performed or not performed any more in the periplasm and thus it emits weak fluorescence. Thus, the unfolded GFP passing through Tat pathway does not folded in the periplasm or the folding process in the periplasm is not effective contrary to the case that unfolded GFP passing through Sec pathway is folded in the periplasm.

Since unfolded GFP by a leader peptide with basic pI value passes through Sec pathway and folded in the periplasm and then emits fluorescence, heterologous proteins passing through Sec pathway and Tat pathway, respectively, are complementary each other regarding whether they have folding mechanisms in the cytosol and in the periplasm, respectively.

Therefore, in order to express a bulky folded active protein in soluble form, when one constitutes a leader peptide with several acidic or basic hydrophilic amino acids linked to Met, 1) proper pI value for the selection of Tat channel, 2) hydrophilicity determining secretion rate, and 3) expression level of the protein (excluding exceptional case of wooble phenomenon) are key factors for soluble expression of the bulky folded active protein thus it is possible to induce soluble expression of the heterologous protein by optimizing the factors properly according to their secretional pathway.

From the examples, the present inventors accomplished the present invention by confirming that soluble expression and secretion of a heterologous protein, particularly a bulky folded active protein which has one or more intrinsic disulfide bonds or transmembrane-like domain is induced by linking a leader peptide with acidic pI and high hydrophilicity thereto; by substituting one or more amino acids within N-terminal of the heterologous protein with ones having acidic or neutral pI and high hydrophilicity; or elevating G_{RNA} value of a polynucleotide encoding the leader peptide having basic pI value and high hydrophilicity.

### INDUSTRIAL APPLICABILITY

The expression vector and the method according to an example of the present invention may be used for the production of recombinant proteins as well as the transduction of therapeutic proteins because it can prevent formation of insoluble inclusion body of a bulky folded heterologous protein having one or more transmembrane-like domains or intramolecular disulfide bonds and enhance secretional efficiency thereof.

### Sequence Listing Free Text

SEQ ID Nos: 1 to 33 are amino acid sequences of modified signal sequences used for expressing of rMefp1 solubly.

SEQ ID Nos: 34 to 66 are nucleotide sequences of forward primers used for cloning expression vectors for expressing the above amino acid sequences as signal sequences.

SEQ ID No: 67 is a nucleotide sequence of a reverse primer used for cloning expression vectors for expressing the above amino acid sequences as signal sequences.

SEQ ID Nos: 68 to 100 are amino acid sequences of various Tat signal sequences.

SEQ ID Nos: 101 to 122 are amino acid sequences of various modified signal sequences of examples of the present invention.

SEQ ID Nos: 123 to 145 are nucleotide sequences of forward primers used for cloning expression vectors for expressing the above modified signal sequences.

SEQ ID No: 146 is a nucleotide sequence of a reverse primer used for cloning expression vectors for expressing the above modified signal sequences.

SEQ ID Nos: 147 to 153 are amino acid sequences of various synthetic signal sequences of examples of the present invention.

SEQ ID No: 154 is an amino acid sequence of Mefp1.

SEQ ID Nos: 155 and 156 are nucleotide sequences of translation initiation regions of pET-22b(+) vector and MKKKKKK-GFP₁₋₅ or MRRRRRR-GFP₁₋₅ encoding regions, respectively.

SEQ ID Nos: 157 and 158 are amino acid sequences of synthetic leader sequences disclosed in Korean Patent Gazette No: 2009-0055457.

While the present invention has been described in connection with certain exemplary examples, it is to be understood that the invention is not limited to the disclosed examples, but, on the contrary, is intended to cover various modifications and equivalent arrangements included within the spirit and scope of the appended claims, and equivalents thereof.

1. An expression vector for enhancing soluble expression and secretion of bulky folded active heterologous proteins having one or more inherent transmembrane-like domains or intramolecular disulfide bonds, comprising a gene construct consisting of:
1) a promoter; and,
2) a polynucleotide operably linked to the promoter, encoding a leader peptide having N-terminal whose pI value is 2.00 to 9.60 and whose hydrophilicity is 1.00 to 2.00.

2. (canceled)

3. The expression vector according to claim 1, wherein the leader peptide is a variant of a signal peptide fragment.

4. The expression vector according to claim 3, wherein the leader peptide further comprises 1 to 30 hydrophilic amino acids linked to carboxy terminal of the variant.

5. The expression vector according to claim 3, wherein the variant is a peptide in which the 2^{nd} and/or the 3^{rd} amino acid of N-terminal of the signal peptide fragment is substituted with aspartate or glutamate.

6. The expression vector according to claim 4, wherein the hydrophilic amino acids is aspartate, glutamate, glutamine, asparagine, threonine, serine, arginine or lysine.

7. The expression vector according to claim 3, wherein the variant consists of 2 to 20 amino acids.

8. The expression vector according to claim 1, wherein the leader peptide is a synthetic peptide consisting of 1 to 30 hydrophilic amino acids linked to carboxy terminal of methionine.

9. The expression vector according to claim 1, wherein the leader peptide is a synthetic peptide consisting of 3 to 16 amino acids linked to carboxy terminal of methionine and at least 60% of the amino acids are hydrophilic.

10.-17. (canceled)

18. A method for enhancing soluble expression and secretion of a bulky folded active heterologous protein having one or more inherent transmembrane-like domains or intramolecular disulfide bonds comprising:
providing a polynucleotide encoding a leader peptide having N-terminal whose pI value is 2.00 to 9.60 and whose hydrophilicity is 1.00 to 2.00;
constructing a gene construct consisting of the polynucleotide and a polynucleotide encoding the bulky folded active heterologous protein having one or more inherent transmembrane-like domains or intramolecular disulfide bonds;
constructing a recombinant expression vector by operably inserting the gene construct into an expression vector;
producing transformants by transforming host cells with the recombinant expression vector; and,
selecting a transformant whose ability for expressing and secreting the bulky folded active heterologous protein is good among the transformants.

19. A method for producing a bulky folded active heterologous protein having one or more inherent transmembrane-like domains or intramolecular disulfide bonds comprising:
providing a polynucleotide encoding a leader peptide having N-terminal whose pI value is 2.00 to 9.60 and whose hydrophilicity is 1.00 to 2.00;
constructing a gene construct encoding a fusion protein sequentially consisting of the leader peptide, a protease recognition site and the bulky folded active heterologous protein having one or more inherent transmembrane-like domains or intramolecular disulfide bonds;
constructing a recombinant expression vector by operably inserting the gene construct into an expression vector;
producing transformants by transforming host cells with the recombinant expression vector;
culturing the transformants by inoculating culture media with the transformants;
isolating the fusion protein; and
isolating a native form of the bulky folded active heterologous protein after cleaving the protease recognition site with a protease is provided.

20. The method according to claim 18, wherein the leader peptide is a variant of a signal peptide fragment.

21. The method according to claim 20, wherein the leader peptide further comprises to 30 hydrophilic amino acids linked to carboxy terminal of the variant.

22. The method according to claim 20, wherein the variant is a peptide in which the 2^{nd} and/or the 3^{rd} amino acid of N-terminal of the signal peptide fragment is substituted with aspartate or glutamate.

23. The method according to claim 21, wherein the hydrophilic amino acids are aspartate, glutamate, glutamine, asparagine, threonine, serine, arginine or lysine.

24. The method according to claim 20, wherein the variant consists of 2 to 20 amino acids.

25. The method according to claim 18, wherein the leader peptide is a synthetic peptide consisting of 1 to 30 hydrophilic amino acids linked to carboxy terminal of methionine.

26. The method according to claim 18, wherein the leader peptide is a synthetic peptide consisting of 3 to 16 amino acids linked to carboxy terminal of methionine and at least 60% of the amino acids are hydrophilic.

27. An expression vector for enhancing soluble expression and secretion of bulky folded active heterologous proteins having one or more inherent transmembrane-like domains or intramolecular disulfide bonds, comprising a gene construct consisting of:
1) a promoter; and,
2) a polynucleotide operably linked to the promoter, encoding a leader peptide having N-terminal whose pI value is 9.90 to 13.35 and whose hydrophilicity is 1.00 to 2.50, wherein the polynucleotide has ΔG_{RNA} value of more than −10.00.

28. (canceled)

29. The expression vector according to claim 27, wherein the leader peptide is a variant of a signal peptide fragment.

30. The expression vector according to claim 29, wherein the leader peptide further comprises to 30 hydrophilic amino acids linked to carboxy terminal of the variant.

31. The expression vector according to claim 29, wherein the variant is a peptide in which the 2^{nd} and/or the 3^{rd} amino acid of N-terminal of the signal peptide fragment is substituted with lysine or arginine.

32. The expression vector according to claim 30, wherein the hydrophilic amino acids are aspartate, glutamate, glutamine, asparagine, threonine, serine, arginine or lysine.

33. The expression vector according to claim 29, wherein the variant consists of 2 to 20 amino acids.

34. The expression vector according to claim 27, wherein the leader peptide is a synthetic peptide consisting of 1 to 30 hydrophilic amino acids linked to carboxy terminal of methionine.

35. The expression vector according to claim 27, wherein the leader peptide is a synthetic peptide consisting of 3 to 16 amino acids linked to carboxy terminal of methionine and at least 60% of the amino acids are hydrophilic.

36. The expression vector according to claim 27, wherein the ΔG_{RNA} value is −7.6 to 1.6.

37.-45. (canceled)

46. A method for enhancing soluble expression and secretion of a bulky folded active heterologous protein having one or more inherent transmembrane-like domains or intramolecular disulfide bonds, the method comprising:
providing a polynucleotide encoding a leader peptide having N-terminal whose pI value is 9.90 to 13.35 and whose hydrophilicity is 1.00 to 2.50, wherein the polynucleotide has ΔG_{RNA} value of more than −10.00;
constructing a gene construct consisting of the polynucleotide and a polynucleotide encoding the bulky folded active heterologous protein having one or more inherent transmembrane-like domains or intramolecular disulfide bonds, wherein the bulky folded active heterologous protein moves into the periplasm as a folded form and has biological activity in periplasm;
constructing a recombinant expression vector by operably inserting the gene construct into an expression vector;
producing transformants by transforming host cells with the recombinant expression vector; and,
selecting a transformant whose ability for expressing and secreting the bulky folded active heterologous protein is good among the transformants.

47. The method according to claim 46, wherein the leader peptide is a variant of a signal peptide fragment.

48. The method according to claim 47, wherein the leader peptide further comprises to 30 hydrophilic amino acids linked to carboxy terminal of the variant.

49. The method according to claim 47, wherein the variant is a peptide in which the 2^{nd} and/or the 3^{rd} amino acid of N-terminal of the signal peptide fragment is substituted with lysine or arginine.

50. The method according to claim 48, wherein the hydrophilic amino acids are aspartate, glutamate, glutamine, asparagine, threonine, serine, arginine or lysine.

51. The method according to claim 47, wherein the variant consists of 2 to 20 amino acids.

52. The method according to claim 46, wherein the leader peptide is a synthetic peptide consisting of 1 to 30 hydrophilic amino acids linked to carboxy terminal of methionine.

53. The method according to claim 46, wherein the leader peptide is a synthetic peptide consisting of 3 to 16 amino acids linked to carboxy terminal of methionine and at least 60% of the amino acids are hydrophilic.

54. The method according to claim 46, wherein the ΔG_{RNA} value is −7.6 to 1.6.

55. The method according to claim 19, wherein the leader peptide is a variant of a signal peptide fragment.

56. The method according to claim 55, wherein the leader peptide further comprises to 30 hydrophilic amino acids linked to carboxy terminal of the variant.

57. The method according to claim 56, wherein the variant is a peptide in which the 2^{nd} and/or the 3^{rd} amino acid of N-terminal of the signal peptide fragment is substituted with aspartate or glutamate.

58. The method according to claim 57, wherein the hydrophilic amino acids are aspartate, glutamate, glutamine, asparagine, threonine, serine, arginine or lysine.

59. The method according to claim 56, wherein the variant consists of 2 to 20 amino acids.

60. The method according to claim 19, wherein the leader peptide is a synthetic peptide consisting of 1 to 30 hydrophilic amino acids linked to carboxy terminal of methionine.

61. The method according to claim 19, wherein the leader peptide is a synthetic peptide consisting of 3 to 16 amino acids linked to carboxy terminal of methionine and at least 60% of the amino acids are hydrophilic.
